SEQUENCE LISTING (24 sequences)

SEQ ID NO: 1 - protein, 56 aa, Homo sapiens
DSLGREAKCYNELNGCTKIYDPVCGTDGNTYPNECVLCFENRKRQTSILIQKSGPC

SEQ ID NO: 2 - protein, 79 aa, Homo sapiens
MKVTGIFLLSALALLSLSGNTGADSLGREAKCYNELNGCTKIYDPVCGTDGNTYPNECVLCFENRKRQTSILIQKSGPC

SEQ ID NO: 3 - protein, 56 aa, Artificial Sequence
  Variant
DSLGREAKCYNELNGCTKIYDPVCGTDGDTYPNECVLCFENRKRQTSILIQKSGPC

SEQ ID NO: 4 - protein, 56 aa, Artificial Sequence
  Variant
DSLGREAKCYNELNGCTRVYDPVCGTDGDTYPNECVLCFENRKRQTSILIQKSGPC

SEQ ID NO: 5 - protein, 56 aa, Artificial Sequence
  Variant
DSLGREAKCYSELNGCTKIYDPVCGTDGNTYPNECVLCFENRKRQTSILIQKSGPC

SEQ ID NO: 6 - protein, 56 aa, Artificial Sequence
  Variant
DSLGREAKCYNELNGCTKIYDPVCGTDGNTYPNECVLCFENQKRQTSILIQKSGPC

SEQ ID NO: 7 - protein, 56 aa, Artificial Sequence
  Variant
DSLGREAKCYNELNGCTKIYDPVCGTDGNTYPNECVLCFENRKSQTSILIQKSGPC

SEQ ID NO: 8 - protein, 56 aa, Artificial Sequence
  Variant
DSLGREAKCYNELNGCTKIYDPVCGTDGNTYPNECVLCFENRTRQTSILIQKSGPC

SEQ ID NO: 9 - protein, 79 aa, Artificial Sequence
  Variant
MKVTGIFLLSALALLSLSGNTGADSLGREAKCYSELNGCTKIYDPVCGTDGNTYPNECVLCFENRKRQTSILIQKSGPC

SEQ ID NO: 10 - protein, 56 aa, Artificial Sequence
  Variant
DSLGREAKCYNELNGCTRIYDPVCGTDGNTYPNECVLCFENRKRQTSILIQKSGPC

SEQ ID NO: 11 - protein, 56 aa, Artificial Sequence
  Variant
DSLGREAKCYNELNGCTHIYDPVCGTDGNTYPNECVLCFENRKRQTSILIQKSGPC

SEQ ID NO: 12 - protein, 56 aa, Artificial Sequence
  Variant
DSLGREAKCYNELNGCTKGYDPVCGTDGNTYPNECVLCFENRKRQTSILIQKSGPC

SEQ ID NO: 13 - protein, 56 aa, Artificial Sequence
  Variant
DSLGREAKCYNELNGCTKAYDPVCGTDGNTYPNECVLCFENRKRQTSILIQKSGPC

SEQ ID NO: 14 - protein, 56 aa, Artificial Sequence
  Variant
DSLGREAKCYNELNGCTKLYDPVCGTDGNTYPNECVLCFENRKRQTSILIQKSGPC

SEQ ID NO: 15 - protein, 56 aa, Artificial Sequence
  Variant
DSLGREAKCYNELNGCTKVYDPVCGTDGNTYPNECVLCFENRKRQTSILIQKSGPC

SEQ ID NO: 16 - protein, 56 aa, Artificial Sequence; identical to SEQ ID NO: 1
  Variant

SEQ ID NO: 17 - protein, 56 aa, Artificial Sequence
  Variant
DSLGREAKCYNELNGCTRGYDPVCGTDGNTYPNECVLCFENRKRQTSILIQKSGPC

SEQ ID NO: 18 - protein, 56 aa, Artificial Sequence
  Variant
DSLGREAKCYNELNGCTRAYDPVCGTDGNTYPNECVLCFENRKRQTSILIQKSGPC

SEQ ID NO: 19 - protein, 56 aa, Artificial Sequence
  Variant
DSLGREAKCYNELNGCTRLYDPVCGTDGNTYPNECVLCFENRKRQTSILIQKSGPC

SEQ ID NO: 20 - protein, 56 aa, Artificial Sequence
  Variant
DSLGREAKCYNELNGCTRVYDPVCGTDGNTYPNECVLCFENRKRQTSILIQKSGPC

SEQ ID NO: 21 - protein, 56 aa, Artificial Sequence
  Variant
DSLGREAKCYNELNGCTHGYDPVCGTDGNTYPNECVLCFENRKRQTSILIQKSGPC

SEQ ID NO: 22 - protein, 56 aa, Artificial Sequence
  Variant
DSLGREAKCYNELNGCTHAYDPVCGTDGNTYPNECVLCFENRKRQTSILIQKSGPC

SEQ ID NO: 23 - protein, 56 aa, Artificial Sequence
  Variant
DSLGREAKCYNELNGCTHLYDPVCGTDGNTYPNECVLCFENRKRQTSILIQKSGPC

SEQ ID NO: 24 - protein, 56 aa, Artificial Sequence
  Variant
DSLGREAKCYNELNGCTHVYDPVCGTDGNTYPNECVLCFENRKRQTSILIQKSGPC

(19) United States
(12) Patent Application Publication
(10) Pub. No.: US 2020/0323954 A1
(43) Pub. Date: Oct. 15, 2020

(54) METHODS AND COMPOSITIONS FOR REDUCING GUT ISCHEMIA/REPERFUSION-INDUCED INJURY

(71) Applicant: PanTheryx, Inc., Boulder, CO (US)

(72) Inventor: Raymond J. Playford, Cobham (GB)

(21) Appl. No.: 16/844,611

(22) Filed: Apr. 9, 2020

Related U.S. Application Data
(60) Provisional application No. 62/831,814, filed on Apr. 10, 2019.

(51) Int. Cl. A61K 38/17; A61P 1/00

(52) U.S. Cl. CPC A61K 38/1709; A61P 1/00

(57) ABSTRACT

Compositions comprising a pancreatic secretory trypsin inhibitor (PSTI) peptide are provided herein for use in reducing hypoxia-reoxygenation-induced cell death, reducing gut ischemia-reperfusion-induced injury, and/or for use in treating or preventing damage to remote organs secondary to gut ischemia-reperfusion.

### CROSS REFERENCES TO RELATED APPLICATIONS

This application claims the benefit of U.S. Provisional Application No. 62/831,814 filed 10 Apr. 2019, the entire contents of which are incorporated herein by reference.

### SEQUENCE LISTING

This application includes a Sequence Listing in electronic format entitled “Sequence-Listing-40735-0047USU1”, which was created on 9 Apr. 2020 and which has a size of 14.6 kilobytes (KB) (14,624 bytes). The contents of txt file “Sequence-Listing-40735-0047USU1” are incorporated by reference herein.

### BACKGROUND OF THE DISCLOSURE

Gastrointestinal ischemia/reperfusion (I/R) injury is involved in multiple clinical situations, such as neonatal necrotizing enterocolitis, acute mesenteric ischemia, volvulus, trauma, cardiopulmonary disease, hemorrhagic shock, intestinal transplant rejection, ischemic colitis, and severe infective colitis. In addition to local tissue injury, remote organs are damaged by the uncontrolled inflammatory response resulting from release of inflammatory mediators and activation of leukocytes due to the post-ischemic gut serving as a priming bed for circulating polymorphonuclear cells. There is also interplay between the inflammatory process and periods of localized tissue hypoxia in conditions such as inflammatory bowel disease where transmigrating neutrophils rapidly deplete the local gut microenvironment of oxygen. (Eltzschig et al. Nat Rev Drug Discov. 2014; 13:852-69). In severe cases, the combination of localized injury with an uncontrolled systemic inflammatory response causes a breakdown in gut mucosal integrity, increased gut permeability and leakage of luminal bacteria and other contents into the circulation. This further exacerbates the injury process, potentially leading to multiple organ failure (MOF) with a mortality rate of up to 80% (Klingensmith et al. Crit Care Clin. 2016; 32:203-12). Current therapeutic options are limited, consisting of general supportive measures in combination with antimicrobials. There is therefore a need for novel therapeutic interventions.

Growth factors, whether produced by purification or using recombinant technology, are increasingly being used for a variety of clinical conditions. Examples include recombinant human insulin, erythropoietin, granulocyte-colony stimulating factor (G-CSF) and interferon. The use of growth factors for ‘hollow organ’ gastrointestinal conditions is, however, at a more preliminary stage.

Pancreatic secretory trypsin inhibitor (PSTI), also known as serine protease inhibitor Kazal type 1 (SPINK1), or tumor associated trypsin inhibitor (TATI), is a 56-amino acid peptide that protects the pancreas from autodigestion because of premature activation of pancreatic proteases. (Kazal et al. J Am Chem Soc. 1948; 70:304-340). Pancreatic secretory trypsin inhibitor (PSTI) has previously been suggested for use in treating pancreatitis.

WO 90/00615, Ohlsson et al., Synergen, Inc., disclose administration of pancreatic secretory trypsin inhibitor (PSTI) for treating or preventing pancreatitis comprising intraductal administration of PSTI, for example, in bile-induced canine pancreatitis model.

US 2006/0068022A1, Playford, Nutritional Bioscience Limited, discloses a method for prophylactically treating a gastrointestinal disorder such as ulcerative colitis by administering an enhanced bioactive agent such as pancreatic secretory trypsin inhibitor. Compositions comprising a bioactive agent, sodium bicarbonate, soy, and licorice are disclosed for treating GI disorders including ulcerative colitis, diverticular disease, and ulcers.

Marchbank et al., 2007, showed pancreatic secretory trypsin inhibitor (PSTI) administration reduces NSAID-induced gastric injury and DSS-induced colitis in rats. PSTI was shown to stimulate cell migration but not proliferation of human colonic carcinoma HT29 cells in vitro. It was also shown that PSTI reduced cytokine release of TNFα and IL-12 in lipopolysaccharide-stimulated dendritic cells in vitro. Marchbank Y, Mahmood A, Fitzgerald A J, Domin J, Butler M, Goodlad R A, Elia G, Cox H M, van Heel D A, Ghosh S, Playford R J, 2007, Am J Pathol, 171 (5), pp. 1462-1473.

Marchbank et al., 2009, showed that that pancreatic secretory trypsin inhibitor (PSTI) reduces NSAID-induced apoptosis. Marchbank T, Weaver G, Nilsen-Hamilton M, Playford R J, Am J Physiol Gastrointest Liver Physiol. 2009 April; 296(4):G697-703. doi: 10.1152/ajpgi.90565.2008. Epub 2009 Jan. 15. Pancreatic secretory trypsin inhibitor is a major motogenic and protective factor in human breast milk.

Marchbank et al., 2013, showed pancreatic secretory trypsin inhibitor (PSTI) causes autocrine-mediated migration and invasion in bladder cancer and phosphorylates the EGF receptor, Akt2 and Akt3, and ERK1 and ERK2. Marchbank T, Mahmood A, Playford R J, Am J Physiol Renal Physiol. 2013 Aug. 1; 305(3):F382-9. doi: 10.1152/ajprenal.00357.2012. Epub 2013 May 22. PSTI was shown to phosphorylate the EGF (epidermal growth factor) receptor and act through various signalling pathways. However, it was also shown that the phosphorylation profile (including timing) was very different to that of EGF.

Previous work showed that PSTI stimulated cells to move (restitution), to dampen down the inflammatory response of stimulated immune cells, and to protect the mucus layer from digestion. However, the mechanisms of ischemia/reperfusion injury are different from NSAID induced or DSS induced colitis.

There is a need for compositions for use in reducing hypoxia-reoxygenation-induced cell death, reducing gut ischemia-reperfusion-induced injury, and for use in treating or preventing damage to remote organs secondary to gut ischemia-reperfusion.

### SUMMARY OF THE INVENTION

The disclosure provides methods for treating, preventing, or alleviating a gastrointestinal ischemia/reperfusion-induced injury or an ischemia/reperfusion-associated condition in a subject in need thereof, comprising administering a composition comprising a therapeutically effective amount of an isolated human pancreatic secretory trypsin inhibitor peptide, a pharmaceutically acceptable salt thereof, or a prodrug thereof, and a pharmaceutically acceptable carrier.

In another embodiment, the disclosure provides compositions for treating, preventing, or alleviating a gastrointestinal ischemia/reperfusion-induced injury or an ischemia/reperfusion-associated condition in a subject in need thereof, the composition comprising a therapeutically effective amount of an isolated human pancreatic secretory trypsin inhibitor peptide, a pharmaceutically acceptable salt thereof, or a prodrug thereof, and a pharmaceutically acceptable carrier.

The ischemia/reperfusion-induced injury may be selected from bacterial translocation, systemic inflammatory response syndrome, intestinal necrosis, intestinal transplant rejection, damage to remote organs, or remote organ failure. The remote organ(s) may be selected from lung, liver, or kidneys, or multiple organ failure.

The ischemia/reperfusion-associated condition may be selected from the group consisting of necrotizing entercolitis, acute mesenteric ischemia, occlusion/infarction, trauma, volvulus, cardiopulmonary disease, transplantation, shock, acute vascular emergency, severe infectious colitis, non-occlusive mesenteric ischemia, ischemic colitis, and intestinal transplantation.

The isolated human pancreatic secretory trypsin inhibitor (PSTI) peptide may be a recombinant or synthetic pancreatic secretory trypsin inhibitor. The PSTI peptide may comprise an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, or a substantially identical variant peptide, or an active peptide fragment or a functional homolog thereof. The PSTI peptide may have an amino acid sequence of SEQ ID NO: 1. The substantially identical variant peptide may share at least 90%, at least 95%, at least 98% or at least 99% sequence identity with SEQ ID NO: 1. The active peptide fragment may consist of at the most 55 consecutive amino acid residues, at the most 50 consecutive amino acid residues, at the most 40 consecutive amino acid residues, at the most 30 consecutive amino acid residues, from 15 to 30 consecutive amino acid residues, or from 18 to 25 consecutive amino acid residues of SEQ ID NO: 1, or a functional homolog thereof. The functional homolog may have at the most three amino acid substitutions, two amino acid substitutions, or one amino acid substitution compared to the active peptide fragment.

The therapeutically effective amount of the PSTI peptide, or pharmaceutically acceptable salt thereof, may be from about 0.001 mg/kg to about 10 mg/kg, about 0.005 mg/kg to about 5 mg/kg, about 0.01 mg/kg to about 1 mg/kg, or about 20 μg/kg to about 200 μg/kg body weight of the subject. The therapeutically effective amount of the PSTI peptide, or pharmaceutically acceptable salt thereof, may be from about 0.01 mg-500 mg, 0.05 mg-70 mg, 0.1-50 mg, 0.5-10 mg, or 1 mg-5 mg per dose. The therapeutically effective amount of the PSTI peptide, or pharmaceutically acceptable salt thereof may vary depending on the route of administration. For example, for administration by enema, the dose of the PSTI peptide, or pharmaceutically acceptable salt thereof, may be selected from a dose within the range of from 50-1000 micrograms per day, optionally in 50-150 ml, or about 100 ml, carrier. For oral administration, the dose of the PSTI peptide, or pharmaceutically acceptable salt thereof, may be from 2-100 mg, or 5-75 mg per day. For systemic treatment, the dose of the PSTI peptide, or pharmaceutically acceptable salt thereof, may be from 10 to 500 micrograms per kg per day, or 20 to 200 micrograms per kg per day.

In a further embodiment, the disclosure provides a method for reducing hypoxia-reoxygenation (H/R)-induced cell death comprising exposing the cell to a composition comprising a PSTI peptide, a pharmaceutically acceptable salt thereof, or a prodrug thereof, and a pharmaceutically acceptable carrier.

The cells may be mammalian cells. The cells may be mammalian gastrointestinal cells. The cells may be human gastrointestinal cells. The mammalian gastrointestinal cells may be human stomach, small intestine, or large intestine cells. The small intestine cells may be duodenum cells, jejunum cells, or ileum cells. The large intestine cells may be cecum, colon, rectal, or anal cells.

In some embodiments, the therapeutically effective amount of the PSTI peptide is an amount that will cause the cell to exhibit one or more, two or more, three or more, four or more, or five or more of the following characteristics when compared to the cell exposed to I/R alone: (i) reduce levels of Caspase 3, (ii) reduce levels of Caspase 9, (iii) reduce levels of Baxα, (iv) normalize Bcl2 levels, (v) cause additional increase in HIF1α above rises caused by I/R alone, (vi) cause additional increase in VEGF above rises caused by I/R alone, (vii) cause additional increase in Hsp70 levels above rises caused by I/R alone, (viii) prevent reduction of tight junction molecule ZO-1, and (ix) prevent reduction of tight junction molecule Claudin1. In some embodiments, the effective amount of the PSTI peptide does not affect increased ICAM1 when compared to the cell exposed to I/R alone.

In some embodiments, the therapeutically effective amount of the PSTI peptide is an amount that will prevent, or alleviate, one or more of bacterial translocation, systemic inflammatory response syndrome, intestinal necrosis, intestinal transplant rejection, damage to remote organs, or remote organ failure in a subject. The remote organs may include lungs, heart, brain, liver, kidneys, or multiple remote organs.

In another embodiment, a composition is provided for use in the manufacture of a medicament for treating, preventing, or alleviating a gastrointestinal ischemia/reperfusion-induced injury or an ischemia/reperfusion-associated condition, the composition comprising a therapeutically effective amount of an isolated human pancreatic secretory trypsin inhibitor peptide, a pharmaceutically acceptable salt thereof, or a prodrug thereof, and a pharmaceutically acceptable carrier.

In a further embodiment, a composition is provided for use in reducing hypoxia-reoxygenation (H/R)-induced cell death comprising exposing the cell to a composition comprising a PSTI peptide, a pharmaceutically acceptable salt thereof, or a prodrug thereof, and a pharmaceutically acceptable carrier.

In some embodiments, a composition comprising a PSTI peptide, a pharmaceutically acceptable salt thereof, or a prodrug thereof, is provided for use in reducing ischemia-reperfusion (I/R)-induced injury.

In some embodiments, a composition comprising a PSTI peptide, a pharmaceutically acceptable salt thereof, or a prodrug thereof, is provided for use in treating or preventing damage to remote organs secondary to gut ischemia-reperfusion.

In some embodiments, a composition is provided for use in manufacture of a medicament for reducing ischemia-reperfusion (I/R)-induced injury, the composition comprising a PSTI peptide, a pharmaceutically acceptable salt thereof, or a prodrug thereof.

In some embodiments, a method is provided for reducing ischemia-reperfusion (I/R)-induced injury in a subject in need thereof, comprising administering a composition comprising a PSTI peptide, a pharmaceutically acceptable salt thereof, or a prodrug thereof to the subject.

In some embodiments, a method is provided for treating, preventing, or alleviating, an ischemia-reperfusion (I/R)-associated condition in a subject in need thereof, comprising administering a composition comprising a PSTI peptide, a pharmaceutically acceptable salt thereof, or a prodrug thereof, to the subject.

The ischemia/reperfusion associated condition may be necrotizing enterocolitis, acute mesenteric ischemia, volvulus, acute vascular emergencies, cardiopulmonary disease, hemorrhagic shock, intestinal transplant rejection, ischemic colitis, severe infective colitis, damage to remote organs, or failure of remote organs.

The ischemia/reperfusion induced injury is selected from the group consisting of bacterial translocation, systemic inflammatory response syndrome, intestinal necrosis, intestinal transplant rejection, damage to remote organs, and remote organ failure.

The composition comprising the PSTI peptide may be administered prior to, concurrent with, or after the subject experiences ischemia/reperfusion associated conditions. The composition comprising the PSTI peptide may be administered prior to, concurrent with, or after, the initial I/R. The composition comprising the PSTI peptide may be administered in a period of time selected from no more than 15 minutes, no more than 30 minutes, no more than 45 minutes, no more than 1 hour, no more than 2 hours, no more than 3 hours, no more than 4 hours, no more than 5 hours, no more than 6 hour, no more than 8 hours, no more than 12 hours, no more than 18 hours, no more than 24 hours, or no more than 36 hours, from the initial ischemia-reperfusion or toxic stress conditions. In some embodiments, the composition comprising the PSTI peptide may be administered to the subject in need thereof up to 24 hours, up to 18 hours, up to 12 hours, up to 8 hours, up to 5 hours, up to 2 hours, up to 1 hour or up to 30 minutes prior to the initial ischemia/reperfusion or toxic stress conditions.

The pancreatic secretory trypsin inhibitor (PSTI) peptide may be a recombinant human PSTI peptide, or a substantially identical variant peptide, active peptide fragment, or functional homolog thereof.

The pancreatic secretory trypsin inhibitor (PSTI) peptide may be an isolated synthetic human PSTI peptide, or a substantially identical variant peptide, active peptide fragment, or functional homolog thereof.

The pancreatic secretory trypsin inhibitor (PSTI) peptide may be an isolated recombinant human PSTI peptide or a pharmaceutically acceptable salt thereof, or prodrug thereof, or a conjugate thereof, having an amino acid sequence of SEQ ID NO: 1, or a substantially identical variant peptide, active peptide fragment, or functional homolog thereof.

The pancreatic secretory trypsin inhibitor (PSTI) peptide may be an isolated recombinant or synthetic human PSTI peptide comprising or having an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, and 24, or a substantially identical variant peptide, active peptide fragment, or functional homolog thereof.

### BRIEF DESCRIPTION OF THE DRAWINGS

FIG. 1A shows effect of hypoxia-reperfusion (H/R) on cell viability in MTT assay in presence and absence of PSTI. AGS cells were exposed to hypoxia (ischemia) and then returned to normoxia (reperfusion). Values are expressed as percentage difference compared to cells incubated in serum free medium alone that were not exposed to H/R (●). Cells incubated in presence of PSTI (10 microgram per ml) but not exposed to H/R showed no change in viability (▾). Results from cells subjected to H/R that were incubated in standard serum free medium (▪) and in the co-presence of various concentrations of PSTI added 1 h prior to H/R (▴) are also shown. ** signifies p<0.01 vs normoxia, $$ signifies p<0.01 vs SFM. Presence of PSTI significantly increased % cell viability of AGS cells when exposed to I/R, compared to cells in SFM.

FIG. 1B shows effect of H/R on cell damage as LDH activity in presence and absence of PSTI. AGS were cells exposed to hypoxia (ischemia) and then returned to normoxia (reperfusion). Values are expressed as percentage difference compared to cells incubated in serum free medium alone that were not exposed to H/R. Cells incubated in presence of PSTI (10 microgram per ml) but not exposed to I/R showed no change in % LDH levels (▾). Results from cells subjected to I/R that were incubated in standard serum free medium (▪) and in the co-presence of various concentrations of PSTI added 1 h prior to H/R (▴) are also shown. ** signifies p<0.01 vs normoxia, $$ signifies p<0.01 vs SFM. Presence of PSTI significantly decreased cell damage of AGS cells when exposed to H/R, compared to cells in SFM.

FIG. 1C shows the effect of treatment with PSTI pre hypoxia, during hypoxia, or post hypoxia in AGS cells when exposed to H/R and assessment as % cell viability in MTT assay. Cells incubated under normoxic conditions throughout (●) was used as a control. Results from cells subjected to H/R that were incubated in standard serum free medium (▪), and in the co-presence of 10 μg/ml of PSTI (▴) are shown. Results shown are for AGS cells, but RIE1 and Caco-2 cells gave similar results (data not shown). ** signifies p<0.01 vs normoxia, $ signifies p<0.05, $$ signifies p<0.01 vs SFM. Presence of PSTI before, or during, H/R significantly increased % cell viability of AGS cells, compared to cells in SFM.

FIG. 1D shows the effect of treatment with PSTI pre hypoxia, during hypoxia, or post hypoxia in AGS cells when exposed to H/R and assessment as cell damage as % LDH activity. Cells incubated under normoxic conditions throughout (●) was used as a control. Results from cells subjected to H/R that were incubated in standard serum free medium (▪) and in the co-presence of 10 μg/ml PSTI (▴) are also shown. Results shown are for AGS cells, but RIE1 and Caco-2 cells gave similar results (data not shown). ** signifies p<0.01 vs normoxia, $$ signifies p<0.01 vs SFM. Presence of PSTI before H/R significantly decreased cell damage of AGS cells, compared to cells in SFM.

FIG. 2A shows effect of PSTI on H/R-induced changes in transepithelial electrical resistance (TEER) in polarised Caco-2 monolayers. Each monolayer had TEER permability assessed at baseline, at the end of 1 h incubation in an hypoxic (ischemic) chamber and at the end of a 24 h reoxygenation (reperfusion) period. Monolayers incubated in serum free medium alone (●) had significantly greater increases in permeability compared to those that had PSTI added to the medium prior to I/R (▴). ** signifies p<0.01 vs baseline normoxia values, $$ signifies p<0.01 vs values seen in cells incubated in medium alone.

FIG. 2B shows effect of PSTI on I/R-induced changes in permeation of HRP through the monolayer. Each monolayer had HRP permeability assessed at baseline, at the end of 1 h incubation in an hypoxic (ischemic) chamber and at the end of a 24 h reoxygenation (reperfusion) period. Monolayers incubated in serum free medium alone (●) had significantly greater increases in permeability compared to those that had PSTI added to the medium prior to I/R (▴). ** signifies p<0.01 vs baseline normoxia values, $$ signifies p<0.01 vs values seen in cells incubated in medium alone.

FIGS. 3A-F show photomicrographs of the effect of PSTI pre-treatment on histology in small intestine (SI) and lung following I/R in mice. Mice underwent a sham (laparotomy only) procedure or subjected to 30 min mesenteric ischemia (I/R) followed by 3 hours of reperfusion. Some animals also received PSTI (20 μg/kg, ip) 1 hour before gut clamping (I/R+PSTI).

FIG. 3A shows a photomicrograph (original magnification 200×) of intestinal tissue from 50% SI region in a sham operated control animal, showing normal histology.

FIG. 3B shows a photomicrograph (original magnification 200×) of intestinal tissue from 50% SI region in an animal that had undergone I/R protocol, showing extensive denudation, necrosis and inflammatory infiltration.

FIG. 3C shows a photomicrograph (original magnification 200×) of intestinal tissue from 50% SI region in an animal that had undergone I/R protocol in an animal pre-treated with PSTI prior to I/R, showing much less extensive damage compared to FIG. 3B without pretreatment.

FIG. 3D shows a photomicrograph of lung tissue (original magnification 400×) from a sham operated control animal, showing essentially normal histology.

FIG. 3E shows a photomicrograph of lung tissue (original magnification 400×) from an animal that had undergone I/R protocol, showing marked pulmonary congestion and diffuse interstitial neutrophilic infiltrates, inflammatory cell infiltrate and areas of tissue destruction.

FIG. 3F shows a photomicrograph of lung tissue (original magnification 400×) from an animal pre-treated with PSTI prior to I/R, showing pre-administration markedly truncated the adverse effects compared to FIG. 3E without pretreatment.

FIGS. 4A-F show effects of I/R+/−PSTI pre-treatment on histological damage, neutrophil accumulation, and lipid peroxidation in an in vivo animal model. Mice underwent a sham (laparotomy only) procedure or subjected to 30 min mesenteric ischemia (I/R) followed by 3 hours of reperfusion. Some animals also received PSTI (20 μg/kg, ip) 1 hour before gut clamping (I/R+PSTI).

FIG. 4A shows a bar graph of the effects of I/R+/−PSTI pre-treatment on histological damage score in small intestine (SI) in an in vivo mouse model. Mice underwent a sham (laparotomy only) procedure or were subjected to 30 min ischemia followed by 3 hours of reperfusion. Some animals also received PSTI (20 μg/kg, ip) 1 hour before gut clamping. Tissue from midportion of clamped SI region (“45-55” SI) was collected, fixed and stained for microscopic scoring. ** signifies p<0.01 vs Sham operated animals, $$ signifies p<0.01 when comparing the effect of PSTI pre-treatment against animals that has undergone I/R without PSTI. PSTI pretreatment significantly reduced histological scoring of small intestine (SI) following I/R.

FIG. 4B shows a bar graph of bar graph of the effects of I/R+/−PSTI pre-treatment on MPO levels in small intestine in an in vivo mouse model. Tissue from midportion of clamped SI region (“45-55” SI, 4A) were collected for analyses of MPO levels. ** signifies p<0.01 vs Sham operated animals, $$ signifies p<0.01 when comparing the effect of PSTI pre-treatment against animals that has undergone I/R without PSTI. PSTI pretreatment significantly reduced MPO levels of 50% small intestine (SI) following I/R.

FIG. 4C shows a bar graph of bar graph of the effects of I/R+/−PSTI pre-treatment on MDA levels in small intestine (SI) in an in vivo mouse model. Tissue from midportion of clamped SI region (“45-55” SI, 4A) were collected for analyses of MDA levels. ** signifies p<0.01 vs Sham operated animals, $$ signifies p<0.01 when comparing the effect of PSTI pre-treatment against animals that has undergone I/R without PSTI. PSTI pretreatment significantly reduced MDA levels of 50% small intestine (SI) following I/R.

FIG. 4D shows a bar graph of the effects of I/R+/−PSTI pre-treatment on histological damage score in lung tissue in an in vivo mouse model. Mice underwent a sham (laparotomy only) procedure or were subjected to 30 min ischemia followed by 3 hours of reperfusion. Some animals also received PSTI (20 μg/kg, ip) 1 hour before gut clamping. Tissue from lung was collected for analysis of histological damage score. ** signifies p<0.01 vs Sham operated animals, $$ signifies p<0.01 when comparing the effect of PSTI pre-treatment against animals that has undergone I/R without PSTI. PSTI pretreatment significantly reduced histological scoring of lung damage compared to animals undergoing I/R who did not receive PSTI.

FIG. 4E shows a bar graph of the effects of I/R+/−PSTI pre-treatment on MPO levels in lung tissue in an in vivo mouse model. Mice underwent a sham (laparotomy only) procedure or were subjected to 30 min ischemia followed by 3 hours of reperfusion. Some animals also received PSTI (20 μg/kg, ip) 1 hour before gut clamping. Tissue from lung was collected for analysis of MPO levels. ** signifies p<0.01 vs Sham operated animals, $$ signifies p<0.01 when comparing the effect of PSTI pre-treatment against animals that has undergone I/R without PSTI. PSTI pretreatment significantly reduced MPO levels in lung tissue following I/R.

FIG. 4F shows a bar graph of bar graph of the effects of I/R+/−PSTI pre-treatment on MDA levels in lung tissue in an in vivo mouse model. Mice underwent a sham (laparotomy only) procedure or were subjected to 30 min ischemia followed by 3 hours of reperfusion. Some animals also received PSTI (20 μg/kg, ip) 1 hour before gut clamping. Tissue from lung was collected for analysis of MDA levels. ** signifies p<0.01 vs Sham operated animals, $$ signifies p<0.01 when comparing the effect of PSTI pre-treatment against animals that has undergone I/R without PSTI. PSTI pretreatment significantly reduced MDA levels in lung tissue following I/R.

FIGS. 5A-5J show effect of PSTI on molecular mechanisms of action in Caco-2 cells in H/R experiments. Caco-2 cells grown in serum free medium exposed to either 1 h or 4 h of hypoxia, followed by 24 h of normoxia (“reperfusion”) (O). Parallel plates treated identically also received PSTI 1 h before undergoing hypoxia+reperfusion (Δ). Changes in protective or damaging pathways are shown. Data expressed as % values found in cells grown in normoxia throughout. * or ** signifies p<0.05 and <0.01 vs normoxic cells. $ and $$ signifies p<0.05 and p<0.01 comparing effect of PSTI on cells grown under same H/R conditions.

FIG. 5A shows a graph of effect of PSTI on Caspase 3 activity in Caco-2 cells in H/R experiments. Caco-2 cells grown in serum free medium exposed to either 1 h or 4 h of hypoxia, followed by 24 h of normoxia (“reperfusion”) (O). Parallel plates treated identically also received PSTI 1 h before undergoing hypoxia+reperfusion (Δ). Changes in damaging pathway Caspase 3 are shown. Cleared cell lysates from H/R experiments in Caco-2 cells were assessed for levels of the apoptotic molecule Caspase 3. ** signifies p<0.01 vs normoxia, $ signifies p<0.05 comparing effect of PSTI pre-treatment against cells that has undergone I/R without PSTI. PSTI pre-treatment significantly decreased Caspase 3 activity in Caco-2 cells after I/R, compared to cells that had undergone H/R without PSTI.

FIG. 5B shows a graph of effect of PSTI on Baxα activity in Caco-2 cells in H/R experiments. Caco-2 cells grown in serum free medium exposed to either 1 h or 4 h of hypoxia, followed by 24 h of normoxia (“reperfusion”) (O). Parallel plates treated identically also received PSTI 1 h before undergoing hypoxia+reperfusion (Δ). Changes in damaging pathways Baxα are shown. Cleared cell lysates from H/R experiments in Caco-2 cells were assessed for levels of the apoptotic molecule Baxα. ** signifies p<0.01 vs normoxia; $ signifies p<0.05, and $$ signifies p<0.01, comparing effect of PSTI pre-treatment against cells that has undergone I/R without PSTI. PSTI pre-treatment significantly decreased Baxα activity in Caco-2 cells after I/R, compared to cells that had undergone H/R without PSTI.

FIG. 5C shows a graph of effect of PSTI on Bcl2 activity in Caco-2 cells in I/R experiments. Caco-2 cells grown in serum free medium exposed to either 1 h or 4 h of hypoxia, followed by 24 h of normoxia (“reperfusion”) (O). Parallel plates treated identically also received PSTI 1 h before undergoing hypoxia+reperfusion (Δ). Changes in protective pathway Bcl2 are shown. Cleared cell lysates from H/R experiments in Caco-2 cells were assessed for levels of the apoptotic molecule Bcl2. * signifies p<0.05, and ** signifies p<0.01 vs normoxia; $ signifies p<0.05, and $$ signifies p<0.01, comparing effect of PSTI pre-treatment against cells that has undergone I/R without PSTI. PSTI pre-treatment significantly attenuated reduction in Bcl2 activity in Caco-2 cells after I/R, compared to cells that had undergone H/R without PSTI

FIG. 5D shows a graph of effect of PSTI on HIF1a activity in Caco-2 cells in H/R experiments. Caco-2 cells grown in serum free medium exposed to either 1 h or 4 h of hypoxia, followed by 24 h of normoxia (“reperfusion”) (O). Parallel plates treated identically also received PSTI 1 h before undergoing hypoxia+reperfusion (Δ). Changes in protective pathway HIF1α are shown. Cleared cell lysates from I/R experiments in Caco-2 cells were assessed for levels of HIF1α. ** signifies p<0.01 vs normoxia; and $$ signifies p<0.01, comparing effect of PSTI pre-treatment against cells that has undergone H/R without PSTI. PSTI pre-treatment significantly increased HIF1α activity in Caco-2 cells after H/R, compared to cells that had undergone H/R without PSTI.

FIG. 5E shows a graph of effect of PSTI on VEGF activity in Caco-2 cells in H/R experiments. Caco-2 cells grown in serum free medium exposed to either 1 h or 4 h of hypoxia, followed by 24 h of normoxia (“reperfusion”) (O). Parallel plates treated identically also received PSTI 1 h before undergoing hypoxia+reperfusion (Δ). Changes in protective pathway VEGF are shown. Cleared cell lysates from I/R experiments in Caco-2 cells were assessed for levels of VEGF. * signifies p<0.05, and ** signifies p<0.01 vs normoxia; $ signifies p<0.05, comparing effect of PSTI pre-treatment against cells that has undergone H/R without PSTI. PSTI pre-treatment significantly increased VEGF activity in Caco-2 cells after H/R, compared to cells that had undergone H/R without PSTI.

FIG. 5F shows a graph of effect of PSTI on hSP70 activity in Caco-2 cells in H/R experiments and under normoxic conditions. Caco-2 cells grown in serum free medium exposed to either 1 h or 4 h of hypoxia, followed by 24 h of normoxia (“reperfusion”) (O). Parallel plates treated identically also received PSTI 1 h before undergoing hypoxia+reperfusion (Δ). Changes in protective pathway hSP70 are shown. Cleared cell lysates from I/R experiments in Caco-2 cells were assessed for levels of hSP70. ** signifies p<0.01 vs normoxia; and $$ signifies p<0.01, comparing effect of PSTI pre-treatment against cells that has undergone H/R without PSTI. PSTI pre-treatment significantly increased hSP70 activity in Caco-2 cells after H/R, compared to cells that had undergone H/R without PSTI. In addition, treatment of Caco-2 cells with PSTI under normoxic conditions also significantly increased hSP70 activity, compared to Caco-2 cells without PSTI under normoxic conditions (data not shown).

FIG. 5G shows a graph of effect of PSTI on ICAM1 activity in Caco-2 cells in H/R experiments. Caco-2 cells grown in serum free medium exposed to either 1 h or 4 h of hypoxia, followed by 24 h of normoxia (“reperfusion”) (O). Parallel plates treated identically also received PSTI 1 h before undergoing hypoxia+reperfusion (Δ). Cleared cell lysates from H/R experiments in Caco-2 cells were assessed for levels of ICAM1. ** signifies p<0.01 vs normoxia. PSTI pre-treatment did not significantly affect ICAM1 activity in Caco-2 cells after H/R, compared to cells that had undergone H/R without PSTI.

FIG. 5H shows a graph of effect of PSTI on ZO1 activity in Caco-2 cells in H/R experiments. Caco-2 cells grown in serum free medium exposed to either 1 h or 4 h of hypoxia, followed by 24 h of normoxia (“reperfusion”) (O). Parallel plates treated identically also received PSTI 1 h before undergoing hypoxia+reperfusion (Δ). Changes in protective pathway ZO1 are shown. Cleared cell lysates from I/R experiments in Caco-2 cells were assessed for levels of the tight junction molecule ZO1. ** signifies p<0.01 vs normoxia; and $$ signifies p<0.01, comparing effect of PSTI pre-treatment against cells that has undergone H/R without PSTI. PSTI pre-treatment significantly attenuated reduction in ZO1 activity in Caco-2 cells after I/R, compared to cells that had undergone H/R without PSTI.

FIG. 5I shows a graph of effect of PSTI on Caspase 9 activity in Caco-2 cells in H/R experiments. Caco-2 cells grown in serum free medium exposed to either 1 h or 4 h of hypoxia, followed by 24 h of normoxia (“reperfusion”) (O). Parallel plates treated identically also received PSTI 1 h before undergoing hypoxia+reperfusion (Δ). Changes in damaging pathway Caspase 9 are shown. Cleared cell lysates from H/R experiments in Caco-2 cells were assessed for levels of the apoptotic molecule Caspase 9. * signifies p<0.05, and ** signifies p<0.01 vs normoxia; $ signifies p<0.05, and $$ signifies p<0.01, comparing effect of PSTI pre-treatment against cells that has undergone H/R without PSTI. PSTI pre-treatment significantly decreased Caspase 9 activity in Caco-2 cells after H/R, compared to cells that had undergone I/R without PSTI.

FIG. 5J shows a graph of effect of PSTI on Claudin1 activity in Caco-2 cells in H/R experiments. Caco-2 cells grown in serum free medium exposed to either 1 h or 4 h of hypoxia, followed by 24 h of normoxia (“reperfusion”) (O). Parallel plates treated identically also received PSTI 1 h before undergoing hypoxia+reperfusion (Δ). Changes in protective pathway Claudin1 are shown. Cleared cell lysates from I/R experiments in Caco-2 cells were assessed for levels of the tight junction molecule Claudin1. ** signifies p<0.01 vs normoxia; and $$ signifies p<0.01, comparing effect of PSTI pre-treatment against cells that has undergone I/R without PSTI. PSTI pre-treatment significantly attenuated reduction in Claudin1 activity in Caco-2 cells after H/R, compared to cells that had undergone H/R without PSTI.

FIG. 6A-6J show effect of PSTI on molecular mechanisms of action in mice at the 50% small intestinal (SI) site that had gut ischemia reperfusion (I/R) injury induced.

FIG. 6A shows a bar graph of the effect of PSTI on Caspase 3 levels at the 50% SI site. Mice had a sham procedure or underwent clamping of the mesenteric vessels for 30 minutes, followed by removal of the clamp and 3 hours of reperfusion. Additional animals underwent the same I/R procedure but had also received PSTI (20 μg/kg, ip) 1 h prior to clamping of mesentery. * or ** signifies p<0.05 and <0.01 vs Sham operated animals, $$ signifies p<0.01 when comparing the effect of PSTI pre-treatment against animals that has undergone I/R without PSTI. PSTI pre-treatment significantly decreased Caspase 3 activity in intestinal tissue after I/R, compared to animals that had undergone I/R without PSTI.

FIG. 6B shows a bar graph of the effect of PSTI on Caspase 9 levels at the 50% SI site. Mice had a sham procedure or underwent clamping of the mesenteric vessels for 30 minutes, followed by removal of the clamp and 3 hours of reperfusion. Additional animals underwent the same I/R procedure but had also received PSTI (20 μg/kg, ip) 1 h prior to clamping of mesentery. * or ** signifies p<0.05 and <0.01 vs Sham operated animals, $$ signifies p<0.01 when comparing the effect of PSTI pre-treatment against animals that has undergone I/R without PSTI. PSTI pre-treatment significantly decreased Caspase 9 activity in intestinal tissue after I/R, compared to animals that had undergone I/R without PSTI.

FIG. 6C shows a bar graph of the effect of PSTI on Baxα levels at the 50% SI site. Mice had a sham procedure or underwent clamping of the mesenteric vessels for 30 minutes, followed by removal of the clamp and 3 hours of reperfusion. Additional animals underwent the same I/R procedure but had also received PSTI (20 μg/kg, ip) 1 h prior to clamping of mesentery. * or ** signifies p<0.05 and <0.01 vs Sham operated animals, $$ signifies p<0.01 when comparing the effect of PSTI pre-treatment against animals that has undergone I/R without PSTI. PSTI pre-treatment significantly decreased the damaging pro-apoptotic Baxα activity in intestinal tissue after I/R, compared to animals that had undergone I/R without PSTI.

FIG. 6D shows a bar graph of the effect of PSTI on Bcl2 levels at the 50% SI site. Mice had a sham procedure or underwent clamping of the mesenteric vessels for 30 minutes, followed by removal of the clamp and 3 hours of reperfusion. Additional animals underwent the same I/R procedure but had also received PSTI (20 μg/kg, ip) 1 h prior to clamping of mesentery. * or ** signifies p<0.05 and <0.01 vs Sham operated animals, $$ signifies p<0.01 when comparing the effect of PSTI pre-treatment against animals that has undergone I/R without PSTI. PSTI pre-treatment significantly increased the protective anti-apoptotic Bcl2 activity in intestinal tissue after I/R, compared to animals that had undergone I/R without PSTI.

FIG. 6E shows a bar graph of the effect of PSTI on HIF1α levels in 50% SI. Mice had a sham procedure or underwent clamping of the mesenteric vessels for 30 minutes, followed by removal of the clamp and 3 hours of reperfusion. Additional animals underwent the same I/R procedure but had also received PSTI (20 μg/kg, ip) 1 h prior to clamping of mesentery. * or ** signifies p<0.05 and <0.01 vs Sham operated animals, $$ signifies p<0.01 when comparing the effect of PSTI pre-treatment against animals that has undergone I/R without PSTI. PSTI pre-treatment significantly increased the protective HIF1α in intestinal tissue after I/R, compared to animals that had undergone I/R without PSTI.

FIG. 6F shows a bar graph of the effect of PSTI on VEGF levels at the 50% SI site. Mice had a sham procedure or underwent clamping of the mesenteric vessels for 30 minutes, followed by removal of the clamp and 3 hours of reperfusion. Additional animals underwent the same I/R procedure but had also received PSTI (20 μg/kg, ip) 1 h prior to clamping of mesentery. * or ** signifies p<0.05 and <0.01 vs Sham operated animals, $$ signifies p<0.01 when comparing the effect of PSTI pre-treatment against animals that has undergone I/R without PSTI. PSTI pre-treatment significantly increased the protective VEGF in intestinal tissue after I/R, compared to animals that had undergone I/R without PSTI

FIG. 6G shows a bar graph of the effect of PSTI on hSP70 levels at the 50% SI site. Mice had a sham procedure or underwent clamping of the mesenteric vessels for 30 minutes, followed by removal of the clamp and 3 hours of reperfusion. Additional animals underwent the same I/R procedure but had also received PSTI (20 μg/kg, ip) 1 h prior to clamping of mesentery. * or ** signifies p<0.05 and <0.01 vs Sham operated animals, $$ signifies p<0.01 when comparing the effect of PSTI pre-treatment against animals that has undergone I/R without PSTI. PSTI pre-treatment significantly increased the protective hSP70 in intestinal tissue after I/R, compared to animals that had undergone I/R without PSTI.

FIG. 6H shows a bar graph of the effect of PSTI on ICAM1 levels at the 50% SI site. Mice had a sham procedure or underwent clamping of the mesenteric vessels for 30 minutes, followed by removal of the clamp and 3 hours of reperfusion. Additional animals underwent the same I/R procedure but had also received PSTI (20 μg/kg, ip) 1 h prior to clamping of mesentery. * or ** signifies p<0.05 and <0.01 vs Sham operated animals, $$ signifies p<0.01 when comparing the effect of PSTI pre-treatment against animals that has undergone I/R without PSTI. PSTI pre-treatment did not affect ICAM1 levels in intestinal tissue after I/R, compared to animals that had undergone I/R without PSTI.

FIG. 6I shows a bar graph of the effect of PSTI on ZO1 levels at the 50% SI site. Mice had a sham procedure or underwent clamping of the mesenteric vessels for 30 minutes, followed by removal of the clamp and 3 hours of reperfusion. Additional animals underwent the same I/R procedure but had also received PSTI (20 μg/kg, ip) 1 h prior to clamping of mesentery. * or ** signifies p<0.05 and <0.01 vs Sham operated animals, $$ signifies p<0.01 when comparing the effect of PSTI pre-treatment against animals that has undergone I/R without PSTI. PSTI pre-treatment significantly increased the protective ZO1 in intestinal tissue after I/R, compared to animals that had undergone I/R without PSTI.

FIG. 6J shows a bar graph of the effect of PSTI on Claudin1 levels at the 50% SI site. Mice had a sham procedure or underwent clamping of the mesenteric vessels for 30 minutes, followed by removal of the clamp and 3 hours of reperfusion. Additional animals underwent the same I/R procedure but had also received PSTI (20 μg/kg, ip) 1 h prior to clamping of mesentery. * or ** signifies p<0.05 and <0.01 vs Sham operated animals, $$ signifies p<0.01 when comparing the effect of PSTI pre-treatment against animals that has undergone I/R without PSTI. PSTI pre-treatment significantly increased the protective Claudin1 in intestinal tissue after I/R, compared to animals that had undergone I/R without PSTI.

FIGS. 7A and B show effect of different times of hypoxia (ischemia) and return to normoxia (reperfusion) on cell viability in a pilot study. Cells were exposed to hypoxia and returned to normoxia for various times in the presence and absence of glucose in the medium during the hypoxic period.

FIG. 7A shows effect of different time exposures to H/R on cell viability measured using MTT assay. Cell were exposed to hypoxia for 1 h (O), 4 h (▪) or 8 h (Δ), and returned to normoxia (reperfusion) for 1, 4, 8 or 24 h in the presence (black symbols) and absence of glucose (open symbols) in the medium during the hypoxic period. Cell viability using MTT assay was then determined. Results shown are for AGS cells, RIE1 and Caco-2 cells gave similar results (data not shown). Cells exposed to hypoxia in the presence of glucose showed no significant change in survival compared to normoxic controls. In contrast, cells incubated in medium without glucose showed significant damage and decreased viability.

FIG. 7B shows effect of different time exposures to H/R on cell damage measured using LDH assay. Cell were exposed to hypoxia for 1 h (O), 4 h (▪) or 8 h (Δ), and returned to normoxia (reperfusion) for 1, 4, 8 or 24 h in the presence (black symbols) and absence of glucose (open symbols) in the medium during the hypoxic period. Cell viability using MTT assay) was then determined. Results shown are for AGS cells, RIE1 and Caco-2 cells gave similar results (data not shown). Cells exposed to hypoxia in the presence of glucose showed no significant change in damage compared to normoxic controls. In contrast, cells incubated in medium without glucose showed significant damage.

### DETAILED DESCRIPTION OF THE INVENTION

Intestinal ischemia/reperfusion (I/R) (also known as gut I/R) occurs during transplantation, mesenteric arterial occlusion, trauma and shock, causing systemic inflammation, multiple organ dysfunction and high mortality. Pancreatic secretory trypsin inhibitor (PSTI) is a serine protease inhibitor, expressed in gut mucosa, whose functions may include mucosal protection and repair. In the present disclosure, it was examined whether PSTI administration affected mesenteric I/R-induced injury. Specifically, viability of AGS, RIE1 and Caco-2 cells exposed to hypoxia/reperfusion (H/R) was determined. Mice underwent ischemia/reperfusion by clamping jejunal section of small intestine and damage was assessed by histological scoring, lipid peroxidation (MDA) and inflammatory infiltration (MPO).

PSTI is a potent serine protease inhibitor initially identified in the pancreas where it helps prevent premature activation of pancreatic proteases. Kazal et al., J Am Chem Soc. 1948, 70:304-340. However, its wider distribution, which includes the epithelium of the normal breast, urothelium, and in the stomach and colon and damaged regions of the small intestine, suggests that it plays additional roles. Marchbank et al. Am J Pathol. 1996; 148:715-722; Freeman et al. Gut. 1990; 31:1318-1323; Playford et al. Am J Pathol. 1995; 146:310-316.

The present inventors previously demonstrated that PSTI stimulates cell migration (restitution), the earliest stage of repair processes and that PSTI administration reduces NSAID-induced gastric injury and DSS-induced colitis in rats. Marchbank et al. Am J Pathol. 2007 November; 171(5):1462-73.

The present disclosure provides compositions comprising PSTI for reducing ischemia/reperfusion (I/R) gut injury, and other conditions involving similar cellular pathways.

### Definitions

The terminology used herein is for the purpose of describing particular embodiments only and is not intended to be limiting of the disclosure.

The singular forms “a”, “an” and “the” are intended to include the plural forms as well, unless the context clearly indicates otherwise.

The term “and/or” refers to and encompasses any and all possible combinations of one or more of the associated listed items.

The term “about,” when referring to a measurable value such as an amount of a compound, dose, time, temperature, and the like, is meant to encompass variations of 10%, 5%, 1%, 0.5%, or even 0.1% of the specified amount.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

All patents, patent applications, and publications referred to herein are incorporated by reference in their entirety.

The embodiments described in one aspect of the present disclosure are not limited to the aspect described. The embodiments may also be applied to a different aspect of the disclosure as long as the embodiments do not prevent these aspects of the disclosure from operating for its intended purpose.

The terms, “patient”, “subject” or “subjects” include but are not limited to humans, the term may also encompass other mammals, or domestic or exotic animals, for example, dogs, cats, ferrets, rabbits, pigs, horses, cattle, birds, or reptiles. In some embodiments, the patient is a human patient. The human patient may be a neonate patient 28 days of age or less, a non-neonate patient older than 28 days, a juvenile patient less than 18 years of age, an adult patient 18 years of age or older, or a senior patient 60 years of age or older.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The “therapeutically effective amount” can vary depending on the compound, the disease and its severity, route of administration, and the condition, age, weight, gender etc. of the subject to be treated.

The terms “treating” or “treatment” of a disease state or condition includes: (i) preventing the disease state or condition, i.e., causing one or more of the clinical symptoms of the disease state or condition not to develop in a subject that may be exposed to or predisposed to the disease state or condition, but does not yet experience or display symptoms of the disease state or condition, (ii) inhibiting or alleviating the disease state or condition, i.e., arresting the development of the disease state or condition or one or more of its clinical symptoms, or (iii) relieving the disease state or condition, i.e., causing temporary or permanent regression of the disease state or condition or one or more of its clinical symptoms.

Abbreviations employed herein include: Baxα; B cell leukemia/lymphoma-2 associated X protein-alpha, Bcl-2; B-cell lymphoma 2, HRP; horse radish peroxidase, H/R; hypoxia/reperfusion, I/R; ischeamia/reperfusion, LDH; lactate dehydroxenase, MDA; malondialdehyde, MPO; myoloperoxidase, MTT; 3-(4,5-dimethylthiazol-2-yl)-2,5-diphenyltetrazolium bromide, PSTI; pancreatic secretory trypsin inhibitor, TEER; transepithelial resistance, TJ; tight junction; ZO1; zona occludens protein.

Pancreatic secretory trypsin inhibitor (PSTI), also known as serine protease inhibitor Kazal type 1 (SPINK1), or tumor associated trypsin inhibitor (TATI), is a 56-amino acid peptide that protects the pancreas from autodigestion due to premature activation of pancreatic proteases. (Kazal et al. J Am Chem Soc. 1948; 70:304-340). PSTI expression also occurs in the normal human breast, in human colostrum (Marchbank et al. Am J Physiol Gastrointest Liver Physiol. 2009; 296:G697-703), in the mucus-producing cells of the mucosa of the stomach and large intestine (Marchbank et al. Am J Pathol. 1996; 148:715-722, Freeman et al. Gut. 1990; 31:1318-1323), and is markedly up-regulated in epithelial and other cells in the small intestine at sites of injury, such as in Crohn's disease, where it is also expressed in the ulcer associated cell lineage (Playford et al. Am J Pathol. 1995; 146:310-316). The present inventors previously have shown that systemic administration of recombinant human PSTI reduces NSAID-induced gastric injury in rats and mice (Marchbank et al. Am J Physiol Gastrointest Liver Physiol. 2009; 296:G697-703, Marchbank et al. Am J Pathol. 2007; 171:1462-73). Taken together, this data suggests that endogenous PSTI may play a protective or healing role against gut injury.

The mechanisms of ischemia/reperfusion injury are different from NSAID induced or DSS induced colitis. Previous work by the present inventors showed that PSTI stimulated cells to move (restitution), to dampen down the inflammatory response of stimulated immune cells, and to protect the mucus layer from digestion. One previous published work focused on receptor activity. Marchbank et al. (2013) showed pancreatic secretory trypsin inhibitor causes autocrine-mediated migration and invasion in bladder cancer and phosphorylates the EGF receptor, Akt2 and Akt3, and ERK1 and ERK2. Marchbank T, Mahmood A, Playford R J, Am J Physiol Renal Physiol. 2013 Aug. 1; 305(3):F382-9. doi: 10.1152/ajprenal.00357.2012. Epub 2013 May 22. PSTI was shown to phosphorylate the EGF (epidermal growth factor) receptor and act through various signalling pathways. However, it was also shown that the phosphorylation profile (including timing) was very different to that of EGF. Another previous published work showed that PSTI reduces NSAID-induced apoptosis. (Marchbank et al., Am J Physiol Gastrointest Liver Physiol. 2009 April; 296(4):G697-703).

However, the damaging pathways involved in intestinal ischemia reperfusion injury are somewhat different than those in NSAID injury or DSS colitis, therefore, it was not at all clear that PSTI would be beneficial in intestinal ischemia reperfusion injury. This is because the damaging pathways involved are not identical. Cells die from necrosis or apoptosis whatever the initiating cause. The various signals that cause this vary according the method of injury used. For example, acid suppressants are helpful for NSAID gastric injury but of no value for inflammatory bowel disease. Prior art has shown that EGF is helpful in ischemia reperfusion injury but the present inventors previous work has shown that while there is some overlap in signaling pathways for PSTI, they are not identical and in particular PSTI does not stimulate cell growth whereas EGF does. It is therefore surprising that PSTI will help in ischemia reperfusion injury. The pathway studies provided herein also demonstrate that PSTI acts through multiple mechanisms of action with respect to apoptosis, VEGF, and cell adhesion molecules which was not known before and are distinct from those caused by other factors such as EGF. As provided herein, the present inventors herein establish that PSTI administration influences gut I/R-induced injury and have examined likely molecular pathways involved in the effects seen.

Pancreatic secretory trypsin inhibitor (PSTI), also known as serine protease inhibitor Kazal type 1 (SPINK1), or tumor associated trypsin inhibitor (TATI), is a 56-amino acid peptide that protects the pancreas from autodigestion due to premature activation of pancreatic proteases. PSTI expression also occurs in the normal human breast, in human colostrum, in the mucus-producing cells of the mucosa of the stomach and large intestine, and is markedly up-regulated in epithelial and other cells in the small intestine at sites of injury, such as in Crohn's disease. The present inventors have previously shown that systemic administration of recombinant human PSTI reduces NSAID-induced gastric injury in rats and mice. Taken together, this data suggests that endogenous PSTI plays a protective or healing role against gut injury.

Some of the earliest pathophysiological processes that occur due to I/R are mucosal epithelial cell damage, increased apoptosis, loss of basement membrane integrity and disruption of barrier function. Unless rapidly repaired, this promotes bacterial and luminal content translocation with induction of local production of cytokines (Hausmann, Int J Inflam. 2010; 2010:574568). To examine this early phase of I/R and MOF injury, a series of in vitro studies was undertaken to examine the effect of hypoxia-normoxia stress on gut integrity in a controlled environment. This involved two well-validated complementary models to examine changes in transepithelial resistance following changes in electrical resistance and passage of a large molecule (HRP) across polarized monolayers of human colonic cancer cells. Davison et al., 2016; Am J Clin Nutr. 2016 August; 104(2):526-36. Although PSTI had no effect on TEER or HRP permeation when added to cells under normoxic conditions, it truncated the damaging effect of hypoxia-normoxia on epithelial integrity by about 65%, mirroring results seen following changes in cell survival.

Having seen positive effects of PSTI in the in vitro models of I/R, studies progressed to an in vivo model. Several different methods are available for inducing gut I/R, for a general review see Gonzalez et al., 2015, Am J Physiol Gastrointest Liver Physiol. 2015; 308(2):G63-75. These models fall into two general categories, the first involves complete temporary arterial occlusion of the anterior mesenteric artery followed by a period of reperfusion (e.g., Berlanga et al., Am J Pathol. 2002; 161(2):373-9), and the second involves segmental mesenteric occlusion by cross clamping the gut causing obstruction of both arteries and veins. The second method was selected because it has the advantages of more closely reproducing the pathophysiology of a section of strangulated bowel, greater reproducibility of injury (Gonzalez et al., 2015), and easy identification of intestine outside of the I/R area that can be used as a control to differentiate local versus systemically mediated injury. As expected, the most obvious damage in the segment where I/R had been induced occurred at the villus tips, with progressive damage extending down the villus length as the amount of injury increased. In contrast, examination of the non-clamped terminal ileal region showed normal histology. Histological examination of lung tissue showed marked acute inflammatory infiltrate with some loss of lung architecture as reported by others (Oguz et al., European Review for Medical and Pharmacological Sciences, 2013; 17: 457-466). Biochemical analyses confirmed the histological findings with markedly raised MPO (neutrophil infiltration) and MDA (lipid peroxidation) levels in affected gut and lung tissue with a smaller but significant rise also seen in kidney tissue. Pre-treatment with PSTI significantly reduced histological and biochemical markers of injury in gut, lung and kidney.

Active caspase-3 (an effector caspase) and active caspase-9 (an initiator caspase) were measured as markers of apoptosis and showed the protocol increased apoptosis 3-6 fold in the in vivo study, with this rise being virtually eliminated if PSTI was pre-administered. PSTI truncated the increase in the pro-apoptotic signalling molecule Baxα and maintained levels of the anti-apoptotic molecule Bcl-2 in the damaged segments, probably contributing to this anti-apoptotic effect. These actions were mediated locally as distant organs and non-clamped regions of bowel did not show any change in caspase, Bcl-2 or Baxα activity. Heat shock proteins maintain cellular homeostasis during normal cell growth and enhance survival during and after cellular stresses that increase accumulation of damaged proteins (Rokutan, J Gastroenterol Hepatol 2000; 15: D12-9) through multiple actions including reducing apoptotic activity. Hsp70 increased in response to PSTI administration when given to cells not subjected to I/R with an even greater rise seen when given to cells exposed to I/R. Similarly, results from whole animal studies showed Hsp70 upregulation in both the clamped and unclamped intestinal segments and in lungs and kidneys. HIF1α and VEGF enhance gut integrity through several mechanisms including reducing apoptosis, stimulating angiogenesis and immune modulation (Cummins et al., Gastroenterology 2008; 134:156-165). HIF1α and VEGF expression increased in response to PSTI in cells exposed to I/R and, in the in vivo model, in the gut region that had undergone FR but not in distant sites. Taken together, the present studies suggest that Hsp70, HIF1α and VEGF may all have relevance to the protective effects of PSTI by reducing apoptosis and through other actions.

ICAM1 is an endothelial- and leukocyte-associated transmembrane protein that stabilizes cell-cell interactions and facilitates leukocyte endothelial transmigration. Its upregulation within the lungs resulting from gut I/R is thought be an important signalling mechanism involved in pulmonary leukocyte infiltration (Zhao et al., 2002, J Appl Physiol. 2002 July; 93(1):338-45). The present studies showed that PSTI reduced neutrophil infiltration of the lungs but did not change the rise in ICAM1 caused by I/R, suggesting that the pulmonary protective effects of PSTI must be through other processes.

Loss of mucosal integrity and increased gut permeability are important exacerbating components of multiple organ failure (MOF) as they allow influx of luminal bacteria and other toxic compounds, resulting in a destructive inflammatory cascade. (Klingensmith et al. Crit Care Clin. 2016; 32:203-12). Intestinal epithelial tight junctions (TJs) are multiprotein complexes that connect adjacent cells on apical and lateral membranes and act as selective barriers. Tight junction integrity is regulated by the assembly of extracellular loops of transmembrane proteins Occludin and Claudin and several intracellular plaque proteins such as ZO1, which link to the actin cytoskeleton. In general terms, the fall in ZO1 and Claudin1 seen in the clamped I/R region but not distant organs, results in lowered intestinal integrity and increased permeability of the affected gut. The truncation of these changes by PSTI administration can be considered potentially beneficial and may have contributed to the beneficial effects seen in the TEER and HRP permeability studies.

The identity of the PSTI receptor(s) involved in these effects is unclear, Niinobu reported ¹²⁵I-labeled PSTI-binding sites on a variety of cell lines, including human skin fibroblasts (BUD-8) and colon cancer (HCT-15) cells. These binding sites were able to be saturated and displaced by excess noniodinated PSTI but not by EGF, were not cell surface proteinases, and the receptor ligand complex had a molecular weight of ˜140 kDa. Niinobu et al., J Exp Med 1990, 172:1133-1142. Previous studies by the present inventors have shown a close relationship between PSTI and the EGF-receptor; the pro-migratory effects of PSTI is inhibited by an EGFR-neutralizing antibody (Marchbank et al., 2013), deletion of EGFR prevents the promigratory activity of PSTI in human colon cancer cells and phosphorylation of EGFR occurs in response to PSTI (Marchbank et al., 2007). Taken together, these results suggest that at least some of the actions of PSTI are mediated through EGFR, although the relationship between PSTI and EGFR is probably not that of a direct receptor ligand but is inducing cross-phosphorylation of EGFR and/or influencing its downstream pathways.

There is always a concern about the use of growth factor therapy in stimulating cancer development in distant organs, especially if given systemically. PSTI has the major potential advantage of stimulating repair without stimulated proliferation when tested against a wide range of cancer cell lines by the present laboratory and other groups (Marchbank et al., 2007). Previous studies by the present inventors examining post receptor signaling showed PSTI causes phosphorylation of AKT1 and ERK and to a lesser extent JNK1, whose functions include influencing apoptosis and the inflammatory response, and RSK1, which is involved in transcriptional regulation by phosphorylating c-Fos and CREB (Marchbank et al., 2013). Without being bound by theory, the present findings probably explain the lack of proliferative activity of PSTI as although it phosphorylates akt2, which is important in cell migration (restitution), it does not cause phosphorylation of akt1 which is involved in stimulating proliferation.

The studies in the present disclosure showed that PSTI protects gut and distant organs from mesenteric I/R-induced injury. Mechanisms involved included reduction in pro-apoptosis signalling, increased HSP70 and VEGF production, and upregulation of tight junction proteins ZO1 and Claudin. Compositions comprising PSTI may therefore provide a novel approach to the prevention and treatment of a wide variety of gut conditions which have I/R as a component of their pathophysiology.

The amino acid sequence of PSTI across species is highly conserved with mouse and human PSTI sharing □61% sequence homology. The present inventors previously showed that the recombinant human PSTI used in the current studies, like the purified form from human pancreas, stimulates migration of human colonic HT29 cells across wounded monolayers. Marchbank et al., 2007, Am J Pathol; 171(5):1462-73. In the present disclosure, it is demonstrated that PSTI administration reduced hypoxia-reoxygenation-induced cell death in human stomach (AGS) and colonic (Caco-2) cells as well as the rat small intestinal cell line RIE1 in a dose and time dependent manner. These results demonstrate wide applicability across the entire gastrointestinal tract and are also not species dependent.

As provided herein, PSTI administration affected mesenteric I/R-induced injury. Specifically, pancreatic secretory trypsin inhibitor is shown herein to reduce multi-organ injury caused by gut ischemia/reperfusion through anti-apoptotic pathways and HIF1α, VEGF, hSP70, ZO1 and Claudin1 modulation.

As provided herein, PSTI reduced I/R induced gut, lung and kidney injury through multiple pathways and may be a useful clinical target to prevent ischemic injury. Specifically, stomach (AGS), intestinal (RIE1) and colonic (Caco-2) cell lines exposed to hypoxia/reperfusion (4 h+24 h) caused 50% drop in viability (MTT assay). PSTI (10 μg/ml) given prior or during hypoxic period improved survival by 50% (p<0.01). Monolayers of polarized Caco-2 cell exposed to hypoxia/reperfusion (1 h+24 h) had 300% increase in transepithelial permeability (horse radish peroxidase translocation). PSTI truncated this by 50% (p<0.01).

As provided herein, adult rats underwent ischemia/reperfusion (30 min, 3 hours) by clamping jejunal section of small intestine causing gut necrosis, lipid peroxidation (MDA) and inflammatory infiltration (MPO). Lung showed significant injury and inflammatory infiltrate with smaller increases in MDA and MPO also seen in kidney. Administration of PSTI (20 μg/kg) reduced all injury markers by 50-80% (p<0.01). In vitro and in vivo studies showed PSTI reduced pro-apototic Caspase 3 and 9 and Baxα levels and normalised Bcl2 levels, caused additional increases in HIF1α, VEGF and Hsp70 levels above rises caused by I/R alone and prevented reduction of tight junction molecules ZO-1 and Claudin1 (all p<0.01). PSTI did not affect increased ICAM1 caused by I/R in gut or lung.

Clinical conditions that can cause intestinal ischmia include neonatal necrotizing entercolitis, acute mesenteric thrombi or mesenteric emboli leading to occlusion/infarction, trauma, volvulus, cardiopulmonary disease or shock leading to non-occlusive mesenteric ischemia, or intestinal transplantation. Intestinal ischemia can lead to bacterial translocation, systemic inflammatory response syndrome, irreversible intestinal necrosis, or remote organ failure. Gonzalez et al., 2015, Am J Physiol Gastrointest Liver Physiol 308:G63-G75.

Methods and compositions comprising a PSTI peptide are provided herein for treating or preventing a gastrointestinal ischemia/reperfusion (I/R)-associated injury. The gastrointestinal ischemia/reperfusion (I/R) associated injury may be associated with neonatal necrotizing enterocolitis, acute mesenteric ischemia, volvulus, trauma, cardiopulmonary disease, hemorrhagic shock, intestinal transplant rejection, ischemic colitis, or severe infectious colitis.

Methods and compositions comprising a PSTI peptide are provided herein for treating or preventing a gastrointestinal ischemia/reperfusion (I/R)-induced injury. The gastrointestinal ischemia/reperfusion (I/R)-induced injury may be bacterial translocation, systemic inflammatory response syndrome, irreversible intestinal necrosis, intestinal transplant rejection, damage to remote organs, or remote organ failure.

Necrotizing enterocolitis is a medical condition where a portion of the bowel dies. About 7% of those born premature may develop necrotizing enterocolitis. Onset is typically within first four weeks of life. Among those affected, about 25% may die. See Necrotizing Enterocolitis-Pediatrics-Merck Manuals Professional Edition February 2017. Necrotizing enterocolitis typically occurs in newborns that are either premature or otherwise unwell. Symptoms include poor feeding, bloating, decreased activity, blood in the stool, or vomiting of bile. The exact cause is unclear. Risk factors include congenital heart disease, birth asphyxia, exchange transfusion, and prolonged rupture of membranes. The underlying mechanism may involve a combination of poor blood flow and infection of the intestines. Diagnosis may be based on symptoms and confirmed with medical imaging. Prevention may include use of breast milk and probiotics. Treatment may include bowel rest, orogastric tube, intravenous fluids, and intravenous antibiotics. Complications may include short-gut syndrome, intestinal strictures, or developmental delay. Nankervis et al., 2008, discloses that based on the demonstration of coagulation necrosis, it is clear that intestinal ischemia plays a role in the pathogenesis of necrotizing enterocolitis (NEC). Intestinal vascular resistance is determined by a dynamic balance between vasoconstrictive and vasodilatory inputs. In the newborn, this balance favors vasodilation secondary to the copious production of endothelium-derived nitric oxide (NO), a circumstance which serves to ensure adequate blood flow and thus oxygen delivery to the rapidly growing intestine. Endothelial cell injury could shift this balance in favor of endothelin (ET)-1-mediated vasoconstriction, leading to intestinal ischemia and tissue injury. Nankervis et al., 2008, Sem Perinatol, 32(2):83-91. In some embodiments, a composition comprising a pancreatic secretory trypsin inhibitor peptide is provided for use in treating or preventing necrotizing enterocolitis. In a particular embodiment, the necrotizing enterocolitis is neonatal necrotizing enterocolitis.

Intestinal ischemia may be classified into three major categories based on its clinical features, namely, acute mesenteric ischemia (AMI), chronic mesenteric ischemia (intestinal angina), and colonic ischemia (ischemic colitis). Acute mesenteric ischemia is not an isolated clinical entity, but a complex of diseases, including acute mesenteric arterial embolus and thrombus, mesenteric venous thrombus, and nonocclusive mesenteric ischemia (NOMI). These diseases have common clinical features caused by impaired blood perfusion to the intestine, bacterial translocation, and systemic inflammatory response syndrome. Reperfusion injury, which exacerbates the ischemic damage of the intestinal microcirculation, is another important feature of AMI. Yasuhara H. Acute mesenteric ischemia: the challenge of gastroenterology. Surg Today 35: 185-195, 2005.

Mesenteric ischemia is a medical condition in which injury to the small intestine occurs due to not enough blood supply. It may occur suddenly, known as acute mesenteric ischemia, or gradually known as chronic mesenteric ischemia. The acute form often presents with sudden severe abdominal pain and is associated with a high risk of death. Risk factors for acute mesenteric ischemia include atrial fibrillation, heart failure, chronic kidney failure, being prone to forming blood clots, and previous myocardial infarction. Diagnosis may be by angiography or computed tomography (CT). Treatment may include stenting, or medications to break down the clot. Most people affected are over 60 years of age. Treatment may be medical or surgical. However, if the bowel has become necrotic, the only treatment is surgical removal of the dead segments of bowel. In some embodiments, a composition comprising a pancreatic secretory trypsin inhibitor peptide is provided for use in treating or preventing an intestinal ischemia. In some embodiments, a composition comprising a pancreatic secretory trypsin inhibitor peptide is provided for use in treating or preventing mesenteric ischemia or ischemic colitis.

Volvulus is a medical condition obstruction caused by abnormal twisting of a portion of the gastrointestinal tract, usually the intestine, which can lead to impaired blood flow and bowel obstruction. Symptoms may include abdominal pain, abdominal bloating, vomiting, constipation, and bloody stool. Diagnosis is typically with medical imaging such as x-rays, a GI series, or CT scan. Regardless of cause, volvulus causes symptoms by bowel instruction manifested as abdominal distension and bilious vomiting, and ischemia (loss of blood flow) to the affected portion of intestine. Volvulus causes severe pain and progressive injury to the intestinal wall, with accumulation of gas and fluid in the portion of bowel obstructed. Ultimately, this can lead to necrosis of the infected intestinal wall, acidosis, and death. Acute volvulus may require immediate surgical intervention to untwist the affected segment of bowel and possibly resect any unsalvageable portion. Volvulus of the small intestine may occur in infants and small children. In infants, volvulus of the small intestine may occur due to malrotation. In adults, volvulus may occur in the colon and is known as a sigmoid volvulus. Causes of sigmoid volvulus may include enlarged colon, abdominal adhesions that develop after surgery, injury or infection, diseases of the large intestine such as Hirschsprung's disease, a colon that is not attached to the abdominal wall, chronic constipation, or pregnancy. Complications of volvulus may include sepsis, or a malabsorption disorder termed short bowel syndrome. See Schwartz M Z. Novel therapies for the management of short bowel syndrome in children. Pediatr Surg Int 29: 967-974, 2013. In some embodiments, a composition comprising a pancreatic secretory trypsin inhibitor peptide is provided for use in treating or preventing complications of volvulus.

Gastrointestinal vascular emergencies include all digestive ischaemic injuries related to acute or chronic vascular and/or haemodynamic diseases. Acute vascular emergencies can arise from direct traumatic injury to the vessel or be spontaneous (non-traumatic). Gastrointestinal ischaemic injuries can be occlusive or non-occlusive, arterial or venous, localized or generalized, superficial or transmural and share the risks of infarction, organ failure and death. Most vascular injuries are life-threatening emergencies since they may cause hypovolemic shock or severe ischemia in the end organ and require prompt diagnosis and treatment. Hypovolemic shock refers to a medical or surgical condition in which rapid fluid loss results in multiple organ failure due to inadequate circulating volume and subsequent inadequate perfusion. Most often, hypovolemic shock is secondary to rapid bleed loss (hemorrhagic shock).

The diagnosis of gastrointestinal ischaemic injuries may be suspected at the initial presentation of any sudden, continuous and unusual abdominal pain, contrasting with normal physical examination. Risk factors are often unknown at presentation and no biomarker is currently available. The diagnosis may be confirmed by ultrasound or preferably by abdominal computed tomography angiography, identifying intestinal ischaemic injury, either with vascular occlusion or in a context of low flow. Multimodal and multidisciplinary management strategies may be employed to avoid large intestinal resections and death. Corcos O, Nuzzo A. Gastrointestinal vascular emergencies. Best Pract Res Clin Gastroenterol 27: 709-725, 2013. In some embodiments, a composition comprising a pancreatic secretory trypsin inhibitor peptide is provided for use in treating or preventing complications of acute vascular emergencies. The acute vascular emergencies may be traumatic gastrointestinal vascular emergencies, and may involve hypovolemic shock, or hemorrhagic shock.

Mesenteric ischaemia is a relatively rare complication following cardiac surgery. Although it is relatively rare (est. 0.49-2.0%) it carries a high risk of mortality (60-100%). Initial symptoms of pain and abdominal distension are not apparent in sedated, ventilated patients. Risk factors for mortality may include age >65 years, poor left ventricular function, recent myocardial infarction within 90 days of surgery, peripheral vascular disease, prolonged bypass time, haemodynamic instability, excessive blood loss, and postoperative atrial fibrillation, standard EuroSCORE, lowest documented blood pressure, and intraoperative vasopressor use. Sastry et al., Interact Cardiovasc Thorac Surg 19(3): 419-424, 2014. In some embodiments, a composition comprising a pancreatic secretory trypsin inhibitor peptide is provided for use in treating or preventing mesenteric ischemia following cardiopulmonary disease or cardiac surgery.

Pancreatic digestive enzymes are released in the ischemic gut during an episode of trauma and hemorrhagic shock may be involved in the generation of distant organ injury. It was previously found that pancreatic duct ligation reduces lung injury following trauma and hemorrhagic shock in a rat model. Cohen et al., 2004 Ann Surg 240 (5):885-891.

Gut and gut-induced lung injury after trauma hemorrhagic shock involves a complex process consisting of intraluminal digestive enzymes, the unstirred mucus layer, and a systemic ischemic-reperfusion injury. Gut and lung injury after trauma shock was prevented by pancreatic enzyme diversion, bolstering the mucus layer and inhibiting mast cell degranulation in a rat model. The study documents the critical balance among the mucus layer, intraluminal digestive enzymes, and mast cells in trauma hemorrhagic shock-induced gut and lung injury. Fishman et al. Ann Surg 260 (6): 1112-1120, 2014.

Ischemia-Reperfusion injury of the intestine is a significant problem in abdominal aortic aneurysm surgery, small bowel transplantation, cardiopulmonary bypass, strangulated hernias, and neonatal necrotizing enterocolitis. It can also occur as a consequence of collapse of systemic circulation, as in hypovolemic and septic shock. It is associated with a high morbidity and mortality. One approach involved ischemic preconditioning, an experimental technique wherein short periods of ischemia are protective against a subsequent ischemic insult. Mallick et al., Dig Dis Sci 49 (9): 1359-1377, 2004. In some embodiments, a composition comprising a pancreatic secretory trypsin inhibitor peptide is provided for use in treating or preventing damage to remote organs caused by trauma, hemorrhagic shock, or intestinal transplant rejection.

Damage to remote organs may be caused by gut ischemia reperfusion. Moore et al. 1994 disclose the postischemic gut serves as a priming bed for circulating neutrophils that provoke multiple organ failure. Moore et al., 1994, J Trauma. 1994 December; 37(6):881-7. Hassoun et al., 2001 investigated post-injury multiple organ failure and the role of the gut. Shock. Hassoun et al., 2001 January; 15 (1):1-10. In some embodiments, a composition comprising a pancreatic secretory trypsin inhibitor peptide is provided for use in treating or preventing damage to remote organs secondary to gut ischemia reperfusion. The damage to remote organs may include damage to lungs, heart, brain, liver, kidneys, or multiple organ failure.

Compositions

The disclosure provides methods and compositions comprising a PSTI peptide and a pharmaceutically-acceptable carrier for treating and/or preventing intestinal ischemia/reperfusion (I/R) injury, necrotizing enterocolitis, acute mesenteric ischemia, volvulus, trauma, cardiopulmonary disease, hemorrhagic shock, intestinal transplant rejection, ischemic colitis, severe infective colitis, or damage to remote organs.

PSTI peptides

Pancreatic secretory trypsin inhibitor (PSTI) is also known as serine protease inhibitor, Kazal-type, 1 (SPINK1), or tumor associated trypsin inhibitor (TATI). The SPINK1 gene encodes pancreatic trypsin inhibitor, which is secreted from acinar cells into pancreatic juice. Its physiologic role was thought to be the prevention of trypsin-catalyzed premature activation of zymogens within the pancreas and the pancreatic duct. Since it has also been found in serum and various normal and malignant tissues. Horii et al., 1987.

Yamamoto et al. (1985) cloned PSTI from a human pancreatic cDNA library. The 79-amino acid sequence contains a 23-amino acid signal peptide followed directly by the 56-amino acid mature secreted form. Northern blot analysis detected PSTI in pancreas and gastric mucosa. Tumor-associated trypsin inhibitor (TATI) is identical to PSTI. TATI is a 6 kD peptide that is synthesized by several tumors and cell lines. It was initially detected in urine of patients with ovarian cancer. This peptide is also produced by the mucosa of the gastrointestinal tract where it may protect the mucosal cells from proteolytic breakdown. Elevated serum and urine levels occur with mucinous ovarian cancer and may occur in nonmalignant diseases such as pancreatitis, severe infections, and tissue destruction. Mutations in the SPINK1 gene have been associated with susceptibility to chronic pancreatitis. These include SPINK1 gene mutations resulting in Asn34Ser, Met1 Thr, Leu14Pro, and Leu14Arg amino acid mutations. The reactive bond for trypsin is position 41-42 of the 79-mer, according to www.uniprot.org/uniprot/P00995. This corresponds to position 18-19 of the mature secreted PSTI peptide 56-mer.

In some embodiments, the isolated human PSTI peptide is a mature secreted form, or a substantially identical variant peptide thereof, functional homolog, or active peptide fragment thereof comprising a consecutive amino acid sequence of said PSTI peptide.

The term fragment is used herein to define any non-full length (as compared to SEQ ID NO: 1) string of amino acid residues that are directly derived from, synthesized to be identical with, or synthesized to have a sequence identity of at least 90% pancreatic secretory trypsin inhibitor peptide comprising the amino acid sequence of SEQ ID NO: 1.

The functional homolog can be defined as comprising the full length amino acid sequence or fragment of mature human PSTI peptide of SEQ ID NO: 1. A functional homolog may be, but is not limited to, a recombinant version of full length of fragmented human PSTI with one or more, two or more, three or more, four or more, or five or more mutations.

The active peptide fragment or functional homolog may be a peptide that exhibits at least some sequence identity with any one of SEQ ID NOs: 1, 2, 3, 4, 5, 6, 7, 8, 9, 10, 11, 12, 13, 14, 15, 16, 17, 18, 19, 20, 21, 22, 23, or 24, and has the ability to induce a statistically significant protective effect in a human colorectal or gastric cell line when placed under H/R conditions in a cell viability (MTT) assay, or lactate dehydrogenase (LDH) assay, compared to the same cell line without exposing to the functional homolog.

The active peptide fragment or functional homolog may be a peptide that exhibits at least some sequence identity with SEQ ID NO: 1, and has the ability to induce a statistically significant protective effect in a human colorectal or gastric cell line when placed under H/R conditions in a cell viability (MTT) assay, or lactate dehydrogenase (LDH) assay, compared to the same cell line without exposing to the functional homolog. In a specific embodiment, the active peptide fragment of the invention consists of at the most 55 amino acid residues, at the most 50 amino acid residues, at the most 40 amino acid residues, at the most 30 amino acid residues, such as 15 to 30 consecutive amino acid residues, or 18 to 25 amino acid residues, of PSTI peptide as identified in SEQ ID NO: 1 or a functional homolog thereof; the functional homolog having at the most three amino acid substitutions, such as two amino acid substitutions, or one amino acid substitution.

In some embodiments, the isolated human PSTI peptide is a protease inhibitory inactivated form in which the reactive site for interacting with the serine proteases comprises an amino acid substitution.

The isolated recombinant PSTI peptide may be produced by any method known in the art. WO 88/03171, Kohno et al., Synergen Biologicals, Inc., discloses preparation of human pancreatic trypsin inhibitors by recombinant methods, for example, in an Escherichia coli host cell.

EP0267692B1, 1994, Ogawa et al., Shionogi & Co. Ltd., disclose a method of production of recombinant human PSTI in Saccharomyces cerevisiae.

U.S. Pat. No. 5,122,594, 1992, Yoshida et al., Shionogi & Co. Ltd., disclose a method of introducing site specific mutations and recombinant production of modified recombinant human PSTI. For example, arginine residues at the 42^{nd} and/or 44^{th} position from the N-terminus of the natural mature human PSTI are replaced by glutamine and/or serine. The modified PSTI peptides are said to have improved stability in terms of decreased susceptibility to decomposition by proteolytic enzymes such as trypsin compared to natural human PSTI.

U.S. Pat. No. 5,126,322, 1992, Collins et al., Bayer A G, disclose a method for producing recombinant human PSTI and variants thereof in an Escherichia coli recombinant host. PSTI variants having various amino acid substitutions at positions 17, 18, 19, 20, 21 and 29 are disclosed.

Marchbank et al., 2007, Am J Physiol 171(5), 1462, discloses production of recombinant human PSTI. A PSTI expression vector was prepared as follows. The hPSTI cDNA sequence was prepared from PANC-1 cells. RNA from these cells was reverse-transcribed and the coding region for PSTI amplified and cloned into the pQE-30 expression vector (Qiagen, Crawley, UK) with an enterokinase cleavage site engineered to allow removal of the polyhistidine tag. The N-terminal histidine-tagged recombinant protein was prepared by expression in transformed IPTG-induced bacteria followed by isolation using nickel-NTA affinity chromatography and enterokinase treatment. Gel electrophoresis, colloidal blue staining, and Western blotting of the final product showed a single band of correct size, and further analysis using mass spectroscopy confirmed the theoretical correct molecular weight (data not shown). The biological activity of recombinant peptide was verified using a trypsin inhibitor assay with Nα-benzoyl-DL-arginine-p-nitroanilide (BAPNA) as substrate (data not shown).

The isolated synthetic PSTI peptide may be produced by any method known in the art. A synthetic PSTI peptide production technique may include a solid phase synthetic peptide protocol and/or a solution phase synthetic peptide protocol.

Optionally the method for preparing the human PSTI peptide may include a protein refolding step. Protein refolding may comprise exposing the PSTI peptide to elevated pressure, e.g., according to PreEMT® technology (Pressure BioSciences, Inc.) that employs high pressure for disaggregation and/or controlled refolding of proteins, e.g., according to methods in U.S. Pat. Nos. 8,710,197, 7,538,198, 7,615,617; US Pub No. 2014/0302589; and/or WO2008/033555.

The isolated human PSTI peptide may comprise a PSTI amino acid sequence as provided herein, or a peptide analog or variant thereof.

Amino acids may be referred to herein by either their commonly known three letter symbols or by the one-letter symbols recommended by the IUPAC-IUB Biochemical Nomenclature Commission. For example, an L-amino acid may be represented herein by its commonly known three letter symbol (e.g., Arg for L-arginine) or by an upper-case one-letter amino acid symbol (e.g., R for L-arginine). A D-amino acid may be represented herein by its commonly known three letter symbol (e.g., D-Arg for D-arginine) or by a lower-case one-letter amino acid symbol (e.g., r for D-arginine).

The term “peptide” refers to a compound made up of a single chain of D- or L-amino acids or a mixture of D- and L-amino acids joined by peptide bonds. Generally, peptides as provided herein are about 15 to about 100 amino acids in length. As non-limiting examples, the integrin-binding peptides present in the conjugates described herein are between about 18 to about 90 amino acids in length, between about 25 to about 85 amino acids in length, between about 50 to about 80 amino acids in length, about 56 amino acids in length, or about 79 amino acids in length.

The term “peptide analog or variant” as used herein refers to a peptide that is comprised of a segment of at least 18 amino acids that has substantial identity to a portion of an PSTI amino acid sequence as provided herein. Typically, peptide analogs or variants comprise a conservative amino acid substitution (or insertion or deletion) with respect to the naturally occurring sequence. Analogs typically are at least 18 amino acids long, at least 20, 30, 40, 50, or 55 amino acids long or longer, and can often be as long as an isolated mature human PSTI peptide 56-mer, or an isolated full-length naturally-occurring human PSTI peptide 79-mer. The variant may comprise a conservative amino acid substitution, or a naturally-occurring amino acid substitution, of one, two, three, four, or more amino acid residues in the PSTI peptide.

Preferred amino acid substitutions are those which: (1) inactivate enzyme activity, (2) reduce susceptibility to proteolysis, (3) reduce susceptibility to oxidation, (4) alter binding affinity for forming protein complexes, (5) alter binding affinities, and (6) confer or modify other physicochemical or functional properties of such analogs. Analogs can include various mutations of a sequence other than the naturally-occurring peptide sequence. For example, single or multiple amino acid substitutions (preferably conservative amino acid substitutions) may be made in the naturally occurring sequence (preferably in the portion of the polypeptide outside the domain(s) forming intermolecular contacts.

Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (▾);

6) Phenylalanine (F), Tyrosine (Y), Tryptophan (W);

7) Serine (S), Threonine (T); and

8) Cysteine (C), Methionine (M).

A conservative amino acid substitution should not substantially change the structural characteristics of the parent sequence (e.g., a replacement amino acid should not tend to break a helix that occurs in the parent sequence, or disrupt other types of secondary structure that characterize the parent sequence). Examples of art-recognized polypeptide secondary and tertiary structures are described in Proteins, Structures and Molecular Principles (Creighton 1984 W. H. Freeman and Company, New York; Introduction to Protein Structure (Branden & Tooze, eds., 1991, Garland Publishing, NY); and Thornton et at. 1991 Nature 354:105, which are each incorporated herein by reference.

As applied to polypeptides, the term “substantial identity” or “substantially identical” means that two peptide sequences, when optimally aligned, such as by the programs GAP or BESTFIT using default gap weights, share at least about 90%, at least about 95%, at least about 98% or at least about 99% sequence identity. Preferably, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; and 6) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-45, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Pearson, 2013, Curr Protoc Bioinformatics, 2013 June; 03: doi:10.1002/0471250953.bi0301s42. Protein analysis software matches similar sequences using measures of similarity assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000), supra). Another preferred algorithm when comparing a sequence of the invention to a database containing a large number of sequences from different organisms is the computer program BLAST, especially blastp or tblastn, using default parameters. See, e.g., Altschul et al. (1990) J. Mol. Biol. 215:403-410 and Altschul et al. (1997) Nucleic Acids Res. 25:3389 402, each of which is herein incorporated by reference. The length of polypeptide sequences compared for homology will generally be at least about 18 amino acid residues, at least about 20 residues, at least about 30 residues, at least about 40 residues, at least about 50 residues, or at least about 55 residues.

The human PSTI peptide may comprise the amino acid sequence of the mature secreted form dslgreakcy nelngctkiy dpvcgtdgnt ypnecvlcfe nrkrqtsili qksgpc (SEQ ID NO: 1), or a substantially identical variant thereof, e.g., a 56-mer peptide having an NCBI ACCESSION 1412241A, e.g., Ogata et al., J. Biol. Chem. 263 (26), 13427-13431 (1988).

The human PSTI peptide may comprise the amino acid sequence of mkvtgiflls alallslsgn tgadslgrea kcynelngct kiydpvcgtd gntypnecvl cfenrkrqts iliqksgpc (SEQ ID NO: 2), e.g., a 79-mer including a 23-mer signal peptide, e.g., having NCBI ACCESSION NP_001341895, or AAA36521, e.g., as disclosed in Yamamoto et al., Biochem. Biophys. Res. Commun. 132 (2), 605-612 (1985).

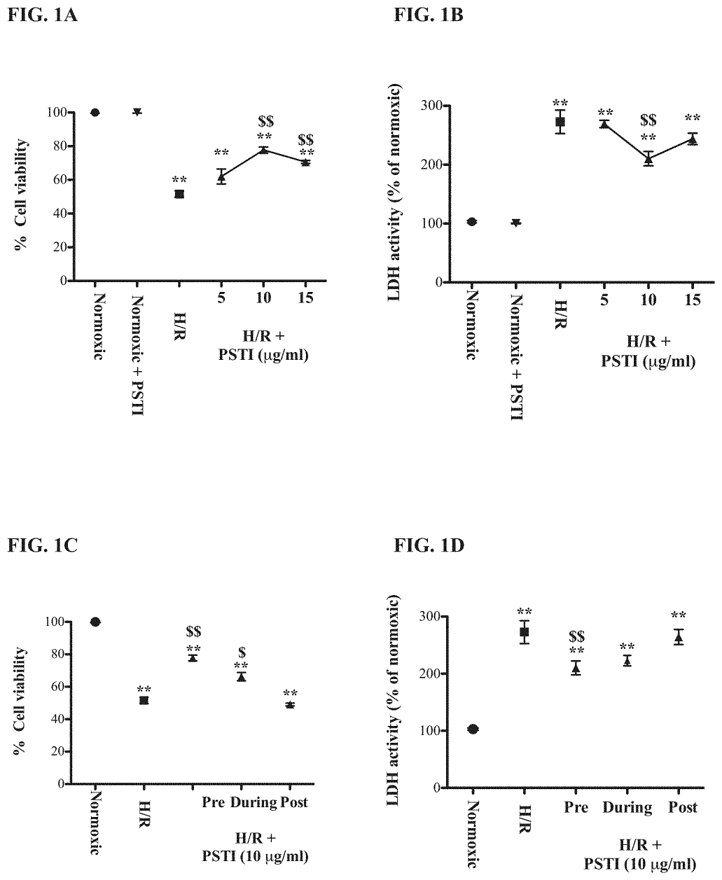

The human PSTI peptide may comprise the amino acid sequence of dslgreakcy nelngctkiy dpvcgtdgdt ypnecvlcfe nrkrqtsili qksgpc (SEQ ID NO: 3), or a substantially identical variant thereof, e.g., a 56-mer peptide, e.g., according to U.S. Pat. No. 5,126,322, FIG. 1.

The human PSTI peptide variant may comprise the amino acid sequence of dslgreakcy nelngctrvy dpvcgtdgdt ypnecvlcfe nrkrqtsili qksgpc (SEQ ID NO: 4), or a substantially identical variant thereof, in which the Lys¹⁸-Ile¹⁹ residues are replaced by Arg¹⁸-Val¹⁹, e.g., (R18/V19) to inactivate the serine protease inhibitor active site, e.g., a 56-mer peptide, e.g., according to Marchbank et al., 2013, Am J Physiol Renal Physiol 305, F382-F389.

The human PSTI peptide variant may comprise, e.g., the amino acid sequence of dslgreakcy selngctkiy dpvcgtdgnt ypnecvlcfe nrkrqtsili qksgpc (SEQ ID NO: 5), or a substantially identical variant thereof, e.g., a 56-mer peptide in which the Asn 11 is replaced with Ser (N11S), e.g., according to Marchbank et al., 2013 Am J Physiol Renal Physiol 305:F382-F389.

The human PSTI peptide may comprise the amino acid sequence of mkvtgiflls alallslsgn tgadslgrea kcyselngct kiydpvcgtd gntypnecvl cfenrkrqts iliqksgpc (SEQ ID NO: 9), e.g., a 79-mer including a 23-mer signal peptide, e.g., having Asn34 is replaced by a Ser (N34S) e.g., having an NCBI accession number AKI71061.1. In some embodiments, the isolated human PSTI peptide comprises an amino acid mutation to reflect a naturally-occurring mutant.

The human PSTI peptide variant may comprise the amino acid sequence of dslgreakcy nelngctkiy dpvcgtdgnt ypnecvlcfe nqkrqtsili qksgpc (SEQ ID NO: 6), or a substantially identical variant thereof, e.g., a 56-mer peptide having a Arg42Gln (R42Q) amino acid substitution, e.g., according to U.S. Pat. No. 5,122,594, 1992, Yoshida et al., e.g., to impart enhanced trypsin stability, e.g., if a longer acting PSTI peptide is desired.

The human PSTI peptide variant may comprise the amino acid sequence of dslgreakcy nelngctkiy dpvcgtdgnt ypnecvlcfe nrksqtsili qksgpc (SEQ ID NO: 7), or a substantially identical variant thereof, e.g., a 56-mer peptide having a Arg44Ser (R44S) amino acid substitution, e.g., according to U.S. Pat. No. 5,122,594, 1992, Yoshida et al, e.g., to impart enhanced trypsin stability e.g., if a longer acting PSTI peptide is desired.

The human PSTI peptide variant may comprise the amino acid sequence of dslgreakcy nelngctkiy dpvcgtdgnt ypnecvlcfe nrtrqtsili qksgpc (SEQ ID NO: 8), or a substantially identical variant thereof, e.g., a 56-mer peptide having a Lys43Thr (K43T) amino acid substitution, e.g., according to U.S. Pat. No. 5,122,594, 1992, Yoshida et al., e.g., to impart decreased trypsin stability, e.g., if a shorter acting PSTI peptide is desired.

The human PSTI peptide may comprise one or two amino acid substitutions of the amino acid sequence of the mature secreted form. For example, the human PSTI peptide may be dslgreakcy nelngctkiy dpvcgtdgnt ypnecvlcfe nrkrqtsili qksgpc (SEQ ID NO: 1), having one or two conservative amino acid substitution(s) selected from the group consisting of K18R, K18H, 119G, 119A, 119L, and 119V. For example, the PSTI peptide may be selected from one or more of SEQ ID NOs: 10, 11, 12, 13, 14, 15, 16, 17, 18, 19, 20, 21, 22, 23, or 24.

Compositions are provided herein comprising an isolated human PSTI peptide and a pharmaceutically acceptable carrier. The isolated PSTI peptide may be an isolated recombinant human PSTI peptide or an isolated synthetic human PSTI peptide, for example, as described herein.

In some embodiments, the isolated human PSTI peptide is provided in the form of a prodrug. As used herein, the term “prodrug” is defined as any compound that undergoes chemical modification before exhibiting its pharmacological effects. For example, the PSTI prodrug may comprise one or more acylated amino acid residues, an amide-bond peptide prodrug, or an ester-based peptide prodrug.

As used herein an “acylated” amino acid is an amino acid comprising an acyl group which is non-native to a naturally-occurring amino acid, regardless by the means by which it is produced. Exemplary methods of producing acylated amino acids and acylated peptides are known in the art and include acylating an amino acid before inclusion in the peptide or peptide synthesis followed by chemical acylation of the peptide. In one embodiment, the acyl group causes the peptide to have one or more of (i) a prolonged half-life in circulation, (ii) a delayed onset of action, (iii) an extended duration of action, and (iv) an improved resistance to proteases.

Prodrug formulations of PSTI peptides analogs are provided wherein the peptide has been modified by linkage of, for example, a hydrophilic group or a dipeptide prodrug element. The prodrugs disclosed herein are presumed to have extended half lives in vivo, and may be converted to the active form at physiological conditions through a non-enzymatic reaction driven by chemical instability. Amide-based peptide prodrugs are disclosed in U.S. Pat. No. 8,946,147. Ester-based peptide prodrugs are disclosed in U.S. Pat. No. 8,697,838.

In some embodiments, the isolated human PSTI peptide is provided in the form of a hydrophilic group peptide conjugate, for example, a PEGylated peptide conjugate. As used herein the term “pegylated” refers to a compound that has been modified from its native state by linking a polyethylene glycol chain to the compound. A “pegylated polypeptide” is a polypeptide that has a PEG chain covalently bound to the polypeptide.

As used herein the general term “polyethylene glycol chain” or “PEG chain”, refers to mixtures of condensation polymers of ethylene oxide and water, in a branched or straight chain, represented by the general formula H(OCH₂CH₂)ₙOH, wherein n is at least 9. Absent any further characterization, the term is intended to include polymers of ethylene glycol with an average total molecular weight selected from the range of 500 to 80,000 Daltons. “Polyethylene glycol chain” or “PEG chain” is used in combination with a numeric suffix to indicate the approximate average molecular weight thereof. For example, PEG-5,000 refers to polyethylene glycol chain having a total molecular weight average of about 5,000 Daltons.

The term “conjugate” refers to a chemical compound that has been formed by the joining or attachment of two or more compounds. In particular, a conjugate of the present invention includes a “PEGylated peptide conjugate” comprising a PSTI peptide covalently attached to a first polyethylene glycol (PEG) moiety at the amino-terminus of the peptide and, optionally, a second PEG moiety at the carboxyl-terminus of the peptide. The conjugate of the present invention may further comprise an imaging agent or a therapeutic agent covalently attached to the peptide, a first PEG moiety, or a second PEG moiety.

PEGylated peptide conjugates may be produced by any method known in the art. For example, see Hamley, Ian W., PEG-Peptide conjugates” Biomacromolecules, 2014, Vol. 15, pp. 1543-1559. PEGylated peptides may be capable of having increased metabolic stability, enhanced water solubility, and/or enhanced retention in one or more selected tissues. For example, a first polyethylene glycol (PEG) moiety may be covalently attached to an amino acid side chain, the amino terminus of the peptide, or the carboxyl-terminus of the peptide. A second polyethylene glycol (PEG) moiety may also be covalently attached to an amino acid side chain, the amino terminus of the peptide, or the carboxyl-terminus of the peptide. In some embodiments, the optional first PEG moiety and the optional second PEG moiety each have a molecular weight of less than about 5000 daltons (Da). In particular embodiments, the first PEG moiety and the second PEG moiety each have a molecular weight of less than about 3000 daltons (Da). In preferred embodiments, the first PEG moiety and the second PEG moiety are monodisperse PEG moieties having a defined chain length.

Compositions are provided comprising an isolated human PSTI peptide and a pharmaceutically acceptable carrier.

As used herein, the term “pharmaceutically acceptable carrier” includes any of the standard pharmaceutical carriers, such as a saline solution, a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The pharmaceutical compositions of the invention may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the invention, provided that the active ingredient in the formulation is not inactivated by the formulation and the formulation is physiologically compatible and tolerable with the route of administration. See also Powell et al. “Compendium of excipients for parenteral formulations” PDA (1998) J Pharm Sci Technol 52:238-311.

The term “pharmaceutical composition” describes a PSTI peptide or pharmaceutically acceptable salt thereof, or prodrug thereof, and one or more pharmaceutically acceptable carriers and optional excipients. The excipient(s) must be acceptable in the sense of being compatible with the other ingredients of the composition and not deleterious to the recipient thereof. In accordance with another aspect of the invention there is also provided a process for the preparation of a pharmaceutical composition including the agent, or pharmaceutically acceptable salts thereof, with one or more pharmaceutically acceptable excipients. The pharmaceutical compositions can be for use in the treatment and/or prophylaxis of any of the conditions described herein. Pharmaceutical compositions adapted for parental administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The terms, “lyophilization”, “lyophilized,” and “freeze-dried” refers to a process by which the material to be dried is first frozen and then the ice or frozen solvent is removed by sublimation in a vacuum environment. The term “lyophilized powder” or “lyophilized preparation” refers to any solid material obtained by lyophilization, i.e., freeze-drying of an aqueous solution. The aqueous solution may contain non-aqueous solvents, i.e. a solution composed of aqueous and one or more non-aqueous solvent(s). Preferably, a lyophilized preparation is one in which the solid material is obtained by freeze-drying a solution composed of water as a pharmaceutically acceptable excipient.

Administration

As used herein, the term “administering” includes oral administration, topical contact, rectal administration, e.g., as a suppository or an enema, intravenous, intraperitoneal, intraductal, intramuscular, intralesional, intrathecal, intranasal, or subcutaneous administration, or the implantation of a slow-release device, e.g., a mini-osmotic pump, to a subject. Administration may be by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, intraductal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering a therapeutically effective amount of a conjugate or composition of the present invention for preventing or relieving one or more symptoms associated with a disease or disorder associated with gut ischemia/reperfusion or an inflammatory condition associated therewith.

The compositions may be administered by any convenient route, for example by infusion or bolus injection, by absorption through epithelial or mucocutaneous linings (e.g., oral mucosa, rectal, and intestinal mucosa, etc.) and may be administered together or co-administered with other biologically active agents.

By “co-administer” it is meant that a PSTI peptide or composition of the present invention may be administered at the same time, just prior to, or just after the administration of a second biologically active agent, e.g., within 48 hours, 36 hours, 24 hours, 18 hours, 12 hours, 6 hours, 3 hours, 1 hour, or within 0.5 hours. For example, the PSTI peptide could be co-administered alongside therapies such as 5-aminosalicylic acid (5ASA) compounds such as mesalazine, sulfasalazine, balsalazide, or olsalazine; coticosteroids such ascortisone, prednisone, hydrocortisone, methylprednisolone, or budesonide; immunosuppressants such as azathioprine, mercaptopurine, or methotrexate; or monoclonal antibodies against immune factors or inflammatory cells, for example, TNF inhibitors such as infliximab, adalimumab, certolizumab pegol, or golimunab; integrin receptor antagonists such as natalizumab, vedolizumab or etrolizumab; an interleukin antagonist such as ustekinumab; or an IL23 specific antagonist such as brazikumab or risankizumab.

The PSTI peptide may also be co-administered or formulated in compositions in combination with nutraceutical agents, for example, in non-parenteral compositions, with bioactive agents such as colostrum, casein, whey, and/or zinc carnosine. The PSTI peptide may be orally administered in site specific release formulations or in combination with other products to reduce its digestion, and enhance stability, such as colostrum, casein, or soya bean trypsin inhibitor.

A pharmaceutical composition of the present invention may be delivered, e.g., subcutaneously, intraperitoneally, or intravenously, with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical compositions of the present invention can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

The pharmaceutical compositions for oral, rectal, or parenteral use described above may be prepared into dosage forms in a unit dose suited to fit a dose of the active ingredients. Such dosage forms in a unit dose include, for example, tablets, pills, capsules, injections (ampoules), suppositories, etc.

Dosing

The PSTI peptide may be administered in an amount of from about 0.001 mg/kg to about 10 mg/kg, about 0.005 mg/kg to about 5 mg/kg, about 0.01 mg/kg to about 1 mg/kg, or about 20-200 μg/kg, or about 20, 25, 30, 40, 45, 50, 60, 70, 80, 90, or 100 μg/kg body weight subject per day, or any value in between. The therapeutically effective amount of the PSTI peptide, or pharmaceutically acceptable salt thereof, may be from about 0.01 mg to about 500 mg, about 0.05 mg to about 80 mg, about 0.1 to about 50 mg, about 0.5 to about 10 mg, or from about 1 mg to about 5 mg per day.

The therapeutically effective amount of the PSTI peptide, or pharmaceutically acceptable salt thereof may vary depending on the route of administration. For example, for administration by enema, the dose of the PSTI peptide, or pharmaceutically acceptable salt thereof, may be selected from a dose within the range of from 50-1000 micrograms per day, optionally in 50-150 ml, or about 100 ml, carrier. For oral administration, the dose of the PSTI peptide, or pharmaceutically acceptable salt thereof, may be from 2-100 mg, or 5-75 mg per day. For systemic treatment, the dose of the PSTI peptide, or pharmaceutically acceptable salt thereof, may be from 10 to 500 micrograms per kg per day, or 20 to 200 micrograms per kg per day.

As provided herein, H/R caused 50% drop in cell viability. PSTI (10 μg/ml) given prior- or during-hypoxic period improved survival by 50% (p<0.01). Caco-2 monolayers exposed to H/R had 300% increase in transepithelial permeability, PSTI truncated this by 50% (p<0.01). Mice that underwent mesenteric I/R had jejunal gut necrosis and 3-fold increases in MDA and MPO. Lung showed similar significant injury and inflammatory infiltrate markers. Smaller increases in MDA and MPO were seen in kidney. PSTI (20 μg/kg) reduced all injury markers by 50-80% (p<0.01). In vitro and in vivo studies showed PSTI reduced pro-apoptotic Caspase 3 and 9 and Baxα levels, normalised Bcl2 levels and caused additional increases in HIF1α, VEGF and Hsp70 above rises caused by I/R alone (all p<0.01). PSTI prevented reduction of tight junction molecules ZO1 and Claudin1 (all p<0.01) but did not affect increased ICAM1 caused by I/R in gut or lung.

### Examples

Materials and Methods

Chemicals were purchased from Sigma (Poole, Dorset) unless otherwise stated. Production of recombinant human PSTI. Recombinant PSTI (SEQ ID NO: 1) was prepared as previously described in Marchbank et al., 2007, Am J Pathol. 2007 November; 171(5):1462-73, and was employed in the following examples.

Cell Lines

Caco-2 is a human cell line derived from colorectal adenocarcinoma of 72-year-old male (ATCC) and exhibits tight junctions and desmosomes between adjacent cells and grows as polarized monolayers (Fogh et al., J Nat'l Cancer Inst 1977; 59:221-6). AGS is a human cell line derived from gastric adenocarcinoma of a 54-year-old female (Barranco et al., 1983, Cancer Res 43, 1703-1709, ATCC). RIE1 is a spontaneously immortalized rat intestinal epithelial cell line (Blay et al., Cell Biol Int Rep 1984 July; 8(7):551-60, ATCC).

Hypoxia (Ischemia)/Normoxia (Reperfusion) (H/R) protocols for in vitro studies: For all experiments AGS, Caco-2 and RIE1 cells were set up in plates as described in specific methods. Cells were used at 70-80% confluence for cell injury/death assessment and as confluent monolayers for permeability studies. To induce “ischemia”, cells were placed in a hypoxic incubator (2% 02) for specified time and to mimic reperfusion, cells were placed back in a normoxic (21% 02) incubator for specified time. All treatments were duplicated in a second set of identical plates under normoxic conditions throughout the same test period as controls. To determine optimum conditions for the in vitro studies, pilot studies were performed.

Cell viability (MTT) assay: Cell viability following H/R was assessed using an MTT assay (3-(4,5-dimethylthiazol-2-yl)-2,5-diphenyltetrazolium bromide) according to Marshall et al., Growth Regul. 1995; 5(2):69-84. The principal of the assay is that viable cells contain NAD(P)H-dependent oxidoreductase enzymes which reduce the MTT reagent to formazan which can be measured spectrophotometrically. At the end of the H/R period, 5 mg/ml MTT in PBS was added to each well of the 96 well plates and incubated at 37° C. for 4 hours, the medium was replaced with 150 μl of DMSO and the absorbance measured at 490 nm. Cell viability was expressed as percentage of the equivalent normoxic control, all results are expressed as mean+/−SEM of six wells.

Lactate dehydrogenase (LDH) assay: Cell damage was assessed using the activity of LDH released into the cell culture medium. LDH is a cytosolic enzyme present in many different cell types. Plasma membrane damage releases LDH into the cell culture media. At the end of the H/R period, supernatant was collected from all wells and LDH activity determined using a Pierce LDH Cytotoxicity Assay Kit (Thermo Fisher Scientific, Hemel Hempstead, UK) following manufacturer's instructions. Results are expressed as LDH activity and presented as mean+/−SEM of six wells.

Pilot Studies

To test the effect of hypoxia (mimicking ischemia-oxygen deprivation) with and without glucose depravation followed by subsequent normoxia (mimicking reperfusion), three different cell lines were set up in 96 well (for cell viability studies) & 24 well (for subsequent permeability studies) plates and subjected to hypoxia for 1, 4 or 8 hours with and without glucose and then were then returned to normoxia in normal glucose containing medium for 0, 1, 4, 8, or 12 hours. Cell Viability (MTT assay) and cell damage (LDH assay) were assessed.

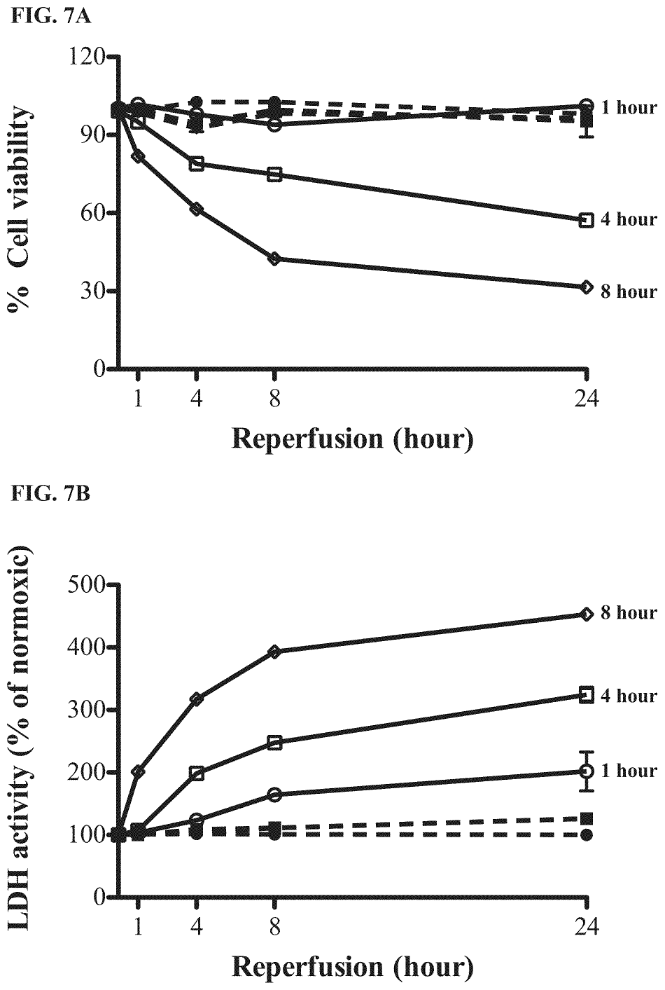

For all three cell lines, it was found that 4 h hypoxia followed by 24 h reperfusion in the absence of glucose gave the optimum conditions for establishing a cell survival rate of approximately 50% (FIG. 7A) with a three-fold increase of LDH (FIG. 7B). Data in FIGS. 7A and B is for AGS cells. For studies examining changes in transepithelial permeability, a protocol of 1 h hypoxia and 24 h reperfusion was used as these conditions resulted in a 30% decrease in permeability but did not affect cell death, allowing a continuous monolayer to be maintained.

### Example 1. Effect of PSTI on Cell Survival

Example 1A: Dose response study: To examine the effect of PSTI on H/R-induced cell injury in vitro, cells were set up in 96 well plates and pre-incubated in SFM containing glucose, with or without 5, 10 or 15 μg/ml PSTI for 1 hour (i.e. PSTI pre-treatment). These concentrations of PSTI were chosen based on previous studies examining cell migration (Marchbank et al., 2007, Am J Pathol 171(5):1462-73). The medium was replaced with SFM without glucose and placed in the hypoxia incubator for 4 hours. At the end of the hypoxia period, medium was replaced with SFM containing glucose for 24 hours at normoxia for the reperfusion period.

Results: 1A: Dose response study: Cells grown under normoxia showed no change in viability or LDH if PSTI was administered. PSTI administered prior to H/R showed PSTI reduced cell death (MTT and LDH assays) with optimum concentration at 10 μg/ml. At this concentration, cell viability significantly increased from 52±2% to 78±2% in the MTT assay (FIG. 1A) and the rise in LDH was significantly reduced by 37%±2% (FIG. 1B).

Example 1B: Efficacy of PSTI given pre-, during- or post-hypoxic period. The potential benefits of giving PSTI prior to, during or following the hypoxic period were compared. Cells were either pre-treated with 10 μg/ml PSTI for the 1 hour before hypoxia (Pre), treated with 10 μg/ml PSTI during hypoxia (During) or treated with 10 μg/ml PSTI during the reperfusion stage (Post). Cell Viability (MTT assay) and cell damage (LDH assay) were assessed. Cells were pre-treated with 10 μg/ml PSTI for the 1 hour before hypoxia (Pre), at the time of the start of hypoxic period (During), or added at the start of the reperfusion stage (Post). Cell Viability (MTT assay) and cell damage (LDH assay) were assessed.

Results: 1B: Efficacy of PSTI given pre-, during- or post-hypoxic period. Studies examining effect of PSTI at 10 μg/ml administered pre-hypoxia, during hypoxia or post-hypoxia (at the reintroduction of normoxia stage) showed that greatest efficacy was seen if PSTI is given pre-hypoxia (FIG. 1C). Similar reciprocal changes were seen following changes in LDH (FIG. 1D). Specifically, cells subjected to H/R without PSTI showed 52±2% viability in MTT assay compared to cells maintained in normoxia, PSTI given pre H/R resulted in a significant increase in viability to 78±2% compared to SFM, PSTI given during hypoxia period resulted in significant increase in viability 66±3% viability and cells given PSTI during the post-hypoxia period showed no benefit, having a cell viability of 49±1% (FIG. 1C). Similar reciprocal changes were seen following changes in LDH (FIG. 1D). Cell Viability (MTT assay) was significantly increased and cell damage (LDH assay) was significantly decreased by exposing cells to PSTI prior to H/R. PSTI administered prior to H/R showed PSTI reduced cell death (MTT and LDH assays).

### Example 2. Effect of PSTI on TEER and Transepithelial Permeability

The effect of 10 μm/ml PSTI pre-treatment on transepithelial permeability induced by 1 hour of hypoxia followed by 24 hours of normoxia was examined using Caco-2, human polarizing colonic adenocarcinoma cells utilizing two different previously published methods according to Davison et al., 2016, Am J Nutr 2016; 104:526-536. One method determined change in transepithelial electrical resistance (TEER) and the other method analyzed the passage of horseradish peroxidase (HRP) across the epithelial layer. HRP (type II) is a non-digestible macromolecular protein (MW=44 kD) and has previously been used as a tracer in evaluation of epithelial permeability. Each monolayer had TEER and HRP permeability assessed at baseline, at the end of 1 h incubation in an hypoxic (ischemic) chamber and at the end of a 24 h reoxygenation (reperfusion) period. Measurements were taken in triplicate from 4 wells per treatment. Results are expressed as mean+/−SEM. The presence of PSTI given prior to H/R had no effect on transepithelial resistance (TEER) under normoxic conditions (data not shown).

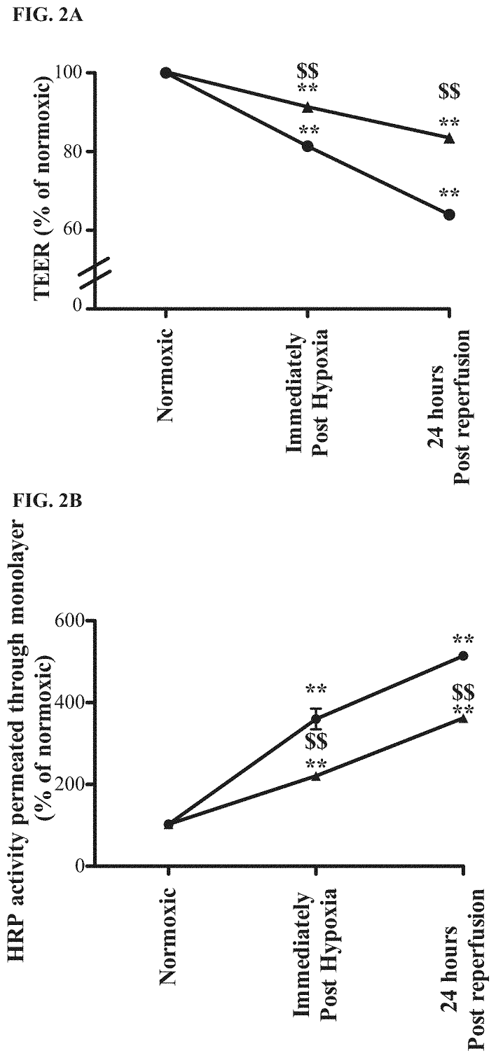

In monolayers subjected to H/R in the absence of PSTI, TEER fell to 81.3±0.28% at the end of the hypoxia period and fell further to 63.9±0.49% at the end of the H/R period. Pre-treatment with PSTI reduced the fall in TEER caused by H/R by about 50% (FIG. 2A). Similar reciprocal results were seen following permeability of HRP across monolayers. Control cells in serum free medium alone had a 3-fold increase in HRP permeability immediately after the hypoxic period, rising to a 5-fold increase at the end of the H/R period. The presence of PSTI truncated the effect of hypoxia by about 54% at the end of the hypoxic period and by 37% at the final H/R time point (FIG. 2B).

### Example 3. Effect of PSTI on Gut and Distant Organ Injury in Response to Small Intestinal I/R

Animal Experiments

All animal experiments were approved by local animal ethics committees and covered by the appropriate licences under the Home Office Animals Procedures Acts, 1986.

I/R model.

C57BL/6 mice (20-25 g) were randomly assigned to one of three groups (n=6-9 per group); a) I/R alone, B) I/R+PSTI (20 μg/kg, ip) treatment 1 hour before the clamping procedure of the I/R protocol, C) Sham group animals, that underwent a laparotomy but no clamping of the SI.

Method of induction of I/R: Mice were anesthetised with ketamine (100 μg/g, ip) and xylazine (10 μg/g, ip) and placed on warming mats. To induce ischemia, the midsection of SI along with its associated mesenteric vessels, comprising approximately the region 30-75% of total length (where entire length is defined as 100%, with proximal duodenum starting at 0% and end of SI as 100% distance) was clamped using non-traumatic surgical clamps (UK Quality Instruments, Ramsgate, Kent) for 30 minutes, followed by removal of the clamp and 3 hours of reperfusion. The wound sites were kept covered and mice kept anesthetised and warm throughout the reperfusion period. Visual inspection of the previously clamped region of the gut was performed at the time of clamp removal to check that the segment became re-perfused.

At the end of the study, mice were killed by cervical dislocation. For intestinal tissues, the position of the various samples was defined by expressing its harvest site as a percentage total length at 50% and 90% small intestinal distance and can be considered as equivalent to jejunum and ileum respectively. The 50% SI collection sample was, therefore, in the middle of the clamped region that had undergone I/R and the 90% site was outside of the previously clamped region. Kidney and lung tissue were also collected. All tissue harvested was split into two, half being snap frozen at −80° C. for further analysis, and the other half immediately fixed in 10% neutral buffered formalin for histopathological assessment.

Histopathological Assessment

Tissue was stained using hemotoxylin-eosin and scored in a blinded manner. Intestinal damage was assessed using the scoring method of Chiu et al. 1970, Arch Surg 101:478 on a scale of 0 (normal) to 5 (damaged severely). 0-Normal mucosa and villi; 1-Development of subepithelial Gruenhagen's space at villus tips, often accompanied by capillary congestion; 2-Extension of subepithelial space with moderate lifting of epithelial layer from lamina propria; 3-Massive lifting down sides of villi, some denuded tips; 4-Denuded villi, with lamina propria and dilated capillaries exposed, increased cellularity of lamina propria may be seen; 5-Digestion and disintegration of the lamina propria, haemorrhage and ulceration.

Lung damage was scored using the method of Koksel et al. 2005, Pharmacol Res; 51:433-462 on a scale of 0 (no damage)-3 (severe damage). 0-no damage; 1-mild neutrophil leukocyte infiltration and mild-moderate interstitial congestion; 2-moderate neutrophil leukocyte infiltration, perivascular edema formation and disintegration of the pulmonary structure; 3-dense neutrophil leukocyte infiltration and absolute destruction of pulmonary structure. Results are expressed as mean+/−SEM.

Tissue Processing for Biochemical Analysis.

Samples were homogenised on ice in ice-cold 50 mM potassium phosphate buffer (pH 6.0) containing 0.5% hexadecyl trimethyl ammonium bromide (HTAB), freeze thawed three times and briefly sonicated. An aliquot of the lysate was stored at −80° C. and the remainder centrifuged at 15,000 rpm for 20 min at 4° C. Supernatants were collected and saved as cleared lysates. Total protein concentration of the lysates and cleared lysates was determined using a standard protein assay.

Myeloperoxidase (MPO) Assay.

MPO activity, used as a marker of neutrophilic infiltration, was measured as described previously (FitzGerald et al. Peptides. 2004 May; 25 (5):793-801). Cleared tissue lysate was incubated with O-dianisidine dihydrochloride (Sigma) and 0.0005% hydrogen peroxide and change in absorbance at 460 nm recorded using a spectrophotometer. One unit of MPO activity was defined as that consuming 1 nmol of peroxide per minute at 22° C. Results are expressed as U/μg of total protein, mean+/−SEM from triplicate wells.

Malondialdehyde (MDA) Assay

MDA levels were determined as a marker of lipid peroxidation from tissue lysates using the thiobarbituric acid method (Satoh K. Clin Chim Acta. 1978 Nov. 15; 90(1):37-43). Results are expressed as nmol MDA/μg of total protein, mean+/−SEM from triplicate wells.

Results: Histological Damage and MPO and MDA Tissue Levels

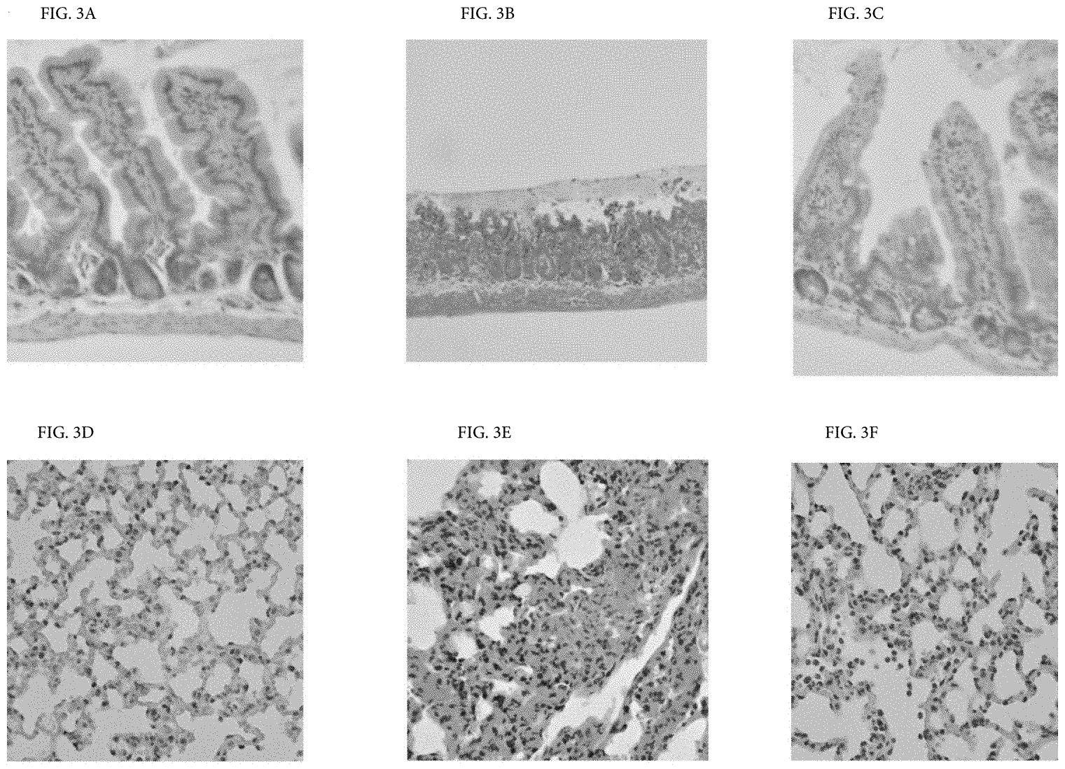

Intestinal tissue from 50% SI region of sham operated control animals showed normal histology (FIG. 3A), whereas the same region of animals that had undergone I/R showed extensive denudation, necrosis and inflammatory infiltration (FIG. 3B). In contrast, animals that had received PSTI prior to I/R showed much less extensive damage (FIG. 3C). Tissue from the 90% SI length (not directly subjected to I/R) appeared histologically normal in all groups (Not shown).

Lung tissue from sham operated control animals had essentially normal histology (FIG. 3D), whereas the I/R group showed marked pulmonary congestion and diffuse interstitial inflammatory cell infiltrate and areas of tissue destruction (FIG. 3E). PSTI pre-administration markedly truncated these effects (FIG. 3F).

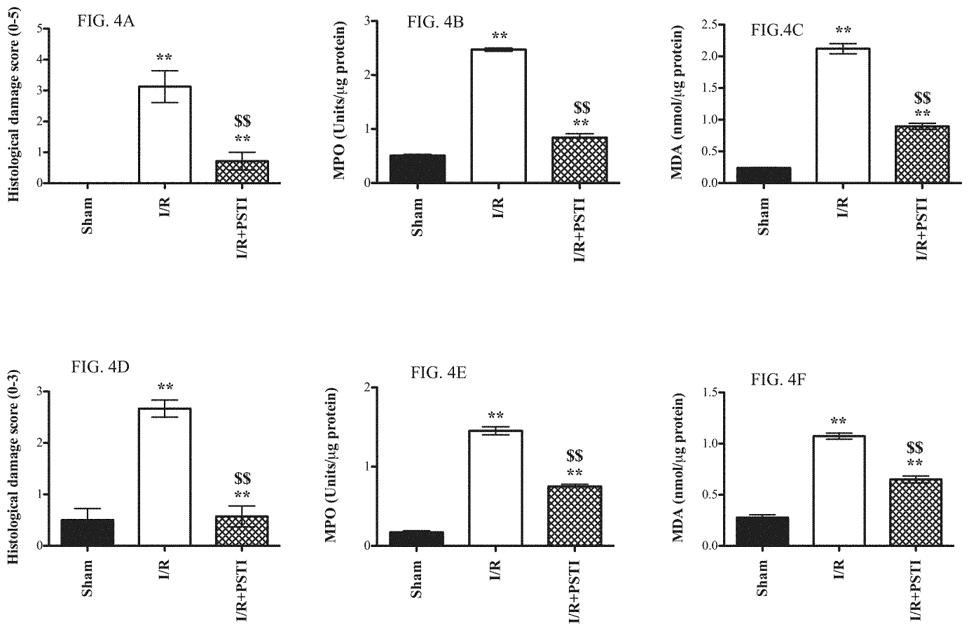

Histological scoring confirmed these protective effects of PSTI in jejunum and lung (FIGS. 4A and 4D). MPO levels were also markedly increased in both 50% SI (FIG. 4B) and lung (FIG. 4E) following I/R with PSTI pre-treatment significantly reducing these changes. Similar results were seen when MDA levels were assessed. In both 50% SI (FIG. 4C) and lung (FIG. 4F) MDA levels were increased in response to I/R and this increase was truncated in animals pre-treated with PSTI. Assessment of tissue from the 90% SI site (not directly subjected to I/R) showed no changes in MPO or MDA (Table 1) following I/R with or without PSTI pre-treatment.

TABLE 1
Effect of gut ischemia-reperfusion (I/R) +/− pre-administration
of PSTI on injury & apoptotic and protective pathways in 90% small
intestine (SI) tissue (non-clamped segment of bowel) site in mice.

| 90% SI | Sham | I/R | I/R + PSTI |
|---|---|---|---|
| Histological score | 0.167 ± 0.167 | 0.375 ± 0.263 | 0.142 ± 0.142 |
| (0-5) | | | |
| MPO | 0.553 ± 0.024 | 0.595 ± 0.031 | 0.582 ± 0.036 |
| (U/μg of protein) | | | |
| MDA | 0.255 ± 0.023 | 0.321 ± 0.035 | 0.251 ± 0.016 |
| (nmol/μg of protein) | | | |
| Caspase 3 | 0.023 ± 0.0014 | 0.023 ± 0.0013 | 0.021 ± 0.007 |
| (Change in absorbance) | | | |
| Caspase 9 | 0.013 ± 0.00044 | 0.014 ± 0.0007 | 0.014 ± 0.00052 |
| (Change in absorbance) | | | |
| Bcl2 | 5.74 ± 0.95 | 5.00 ± 0.83 | 5.55 ± 1.00 |
| (pg/μg total protein) | | | |
| Baxα | 3.38 ± 0.22 | 3.35 ± 0.24 | 2.13 ± 0.11 ** $$ |
| (pg/μg total protein) | | | |
| HIF1α | 0.73 ± 0.1 | 0.75 ± 0.05 | 0.7 ± 0.12 |
| (pg/μg total protein) | | | |
| VEGF | 1.58 ± 0.08 | 1.54 ± 0.04 | 1.65 ± 0.09 |
| (pg/μg total protein)) | | | |
| HSP70 | 18.5 ± 0.41 | 18.7 ± 0.28 | 21.65 ± 0.35 ** $$ |
| (pg/μg total protein) | | | |
| ICAM1 | 2.17 ± 0.026 | 1.62 ± 0.35 | 1.88 ± .12 |
| ((pg/μg total protein) | | | |
| ZO1 | 0.172 ± 0.8 | 0.158 ± 0.008 | 0.17 ± 0.008 |
| (pg/μg total protein) | | | |
| Claudin 1 | 0.158 ± 0.014 | 0.15 ± 0.008 | 0.152 ± 0.014 |
| (pg/μg total protein) | | | |

Most parameters remained normal at the non-clamped region. Data presented as mean +/− SEM.
** signifies p < 0.05 and <0.01 vs Sham operated animals (laparotomy only),
$$ signifies p < 0.05 and <0.01 vs I/R.

Table 1 shows that apoptotic molecule Baxα is significantly decreased in 90% small intestine (90% SI) in non-clamped bowel segment in mouse in vivo model after ischemia-reperfusion when pre-treated with PSTI compared to either sham operated animals, or animals subjected to I/R without pre-treatment.

Table 1 also shows that heat shock protein 70 (hSP70) is significantly increased in 90% small intestine (90% SI) in non-clamped bowel segment in mouse in vivo model after ischemia-reperfusion when pre-treated with PSTI compared to either sham operated animals, or animals subjected to I/R without pre-treatment.

Histological examination of renal tissue showed minimal changes although biochemical assessment showed a small but significant increase in both MDA and MPO levels following I/R (Table 2).

TABLE 2
Effect of gut ischemia-reperfusion (I/R) +/− pre-administration
of PSTI on injury & apoptotic and protective pathways in renal tissue in mice.

| KIDNEY | Sham | I/R | I/R + PSTI |
|---|---|---|---|
| MPO | 0.157 ± 0.013 | 0.509 ± 0.048 ** | 0.291 ± 0.009 ** $$ |
| (U/μg of protein) | | | |
| MDA | 0.29 ± 0.023 | 0.55 ± 0.049 ** | 0.47 ± 0.047 ** |
| (nmol/μg of protein) | | | |
| Caspase 3 activity | 0.073 ± 0.017 | 0.089 ± 0.019 | 0.069 ± 0.019 |
| (Change in absorbance) | | | |
| Caspase 9 activity | 0.042 ± 0.005 | 0.049 ± 0.005 | 0.053 ± 0.010 |
| (Change in absorbance) | | | |
| Bcl2 | 67.89 ± 7.93 | 65.34 ± 5.21 | 66.37 ± 4.89 |
| (pg/μg total protein) | | | |
| Baxα | 5.59 ± 0.88 | 4.81 ± 0.37 | 5.61 ± 0.63 |
| (pg/μg total protein) | | | |
| HIF1α | 7.46 ± 0.52 | 7.46 ± 1.27 | 9.45 ± 1.09 |
| (pg/μg total protein) | | | |
| VEGF | 2.69 ± 0.03 | 2.31 ± 0.2 | 2.53 ± 0.19 |
| (pg/μg total protein) | | | |
| hSP70 | 23.6 ± 1.02 | 34.1 ± 1.11 ** | 36.6 ± 2.96 ** |
| (pg/μg total protein) | | | |
| ICAM1 | 5.1 ± 0.14 | 4.9 ± 0.16 | 5.22 ± 0.42 |
| ((pg/μg total protein) | | | |
| ZO1 | 0.154 ± 0.008 | 0.144 ± 0.006 | 0.151 ± 0.008 |
| (pg/μg total protein) | | | |
| Claudin 1 | 0.15 ± 0.04 | 0.13 ± 0.002 | 0.154 ± 0.016 |
| (pg/μg total protein) | | | |

Data presented as mean +/− SEM.
** signifies p < 0.05 and <0.01 vs Sham operated animals (laparotomy only),
$$ signifies p < 0.05 and <0.01 vs I/R.

Table 2 shows renal MPO levels were significantly decreased in mouse in vivo model following I/R when pre-treated with PSTI, compared to rats subjected to I/R without PSTI pre-treatment (Table 2).

### Example 4. Mechanisms of Action of PSTI in the In Vitro and In Vivo Studies

Having shown that PSTI reduced cell and organ damage in our in vitro and in vivo studies, samples from cell viability and whole animal I/R tissues were further analysed to examine PSTI's possible modes of action.

Cell apoptosis assays: Active caspase-3 and active caspase-9 were determined using methods described previously (Davison et al., Am J Clin Nutr 2016 August; 104(2):526-36, using commercial colorimetric assay kits (BF3100 and BF10100, R&D Systems). Concentrations of the anti-apoptotic protein Bcl2 and the pro-apoptotic protein Baxα were determined in the same cell lysates as used for caspase analyses, using Duoset Elisa kits (R&D Systems Europe Ltd).

Measurements of HIF1α, VEGF, hSP70 and ICAM-1: HIF1α, VEGF, hSP70 and ICAM1 concentration in the cleared cell lysates was determined using Duoset Elisa kits as per the manufacturer's instructions (R&D Systems Europe). hSP70 concentrations were determined using the present inventor's previously published methods (Marchbank et al. Am J Physiol Gastrointest Liver Physiol. 2011; 300:G477-84) with a Duoset Elisa kit (R&D Systems Europe).

Tight Junction proteins: ZO1 and Claudin-1 concentrations were determined using previously published methods (Davison et al. Am J Clin Nutr. 2016 August; 104(2):526-36) and standard ELISA kits (tight junction antibody samples pack 90-1200, Invitrogen)

Statistical Analyses

All values are expressed as the mean+/−SEM unless otherwise stated. Data were analysed using a one way ANOVA. Where a significant effect was seen (p<0.05), individual comparisons were performed using t-tests based on the group means, residual and degrees of freedom obtained from the ANOVA, a method equivalent to repeated measures analyses.

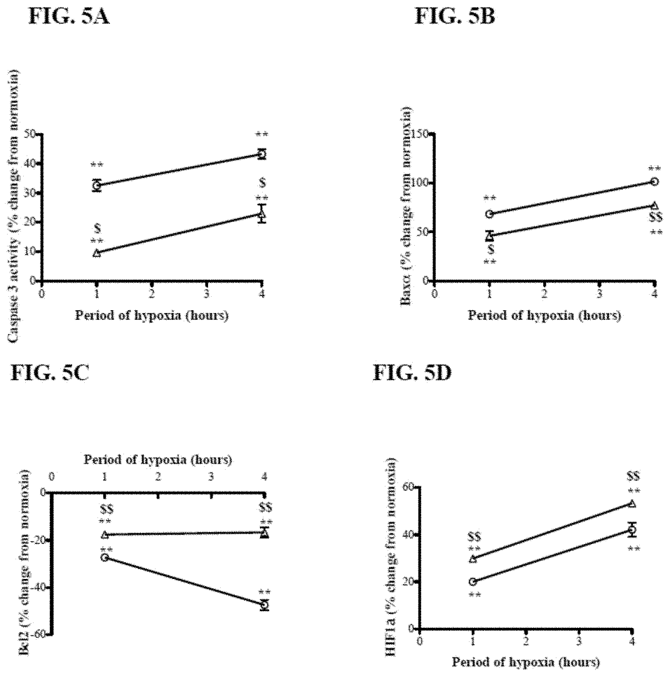

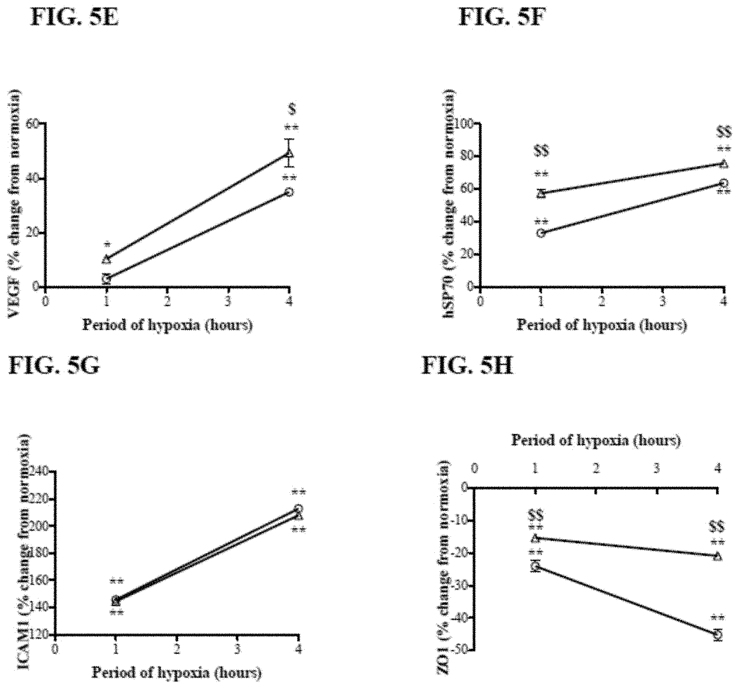

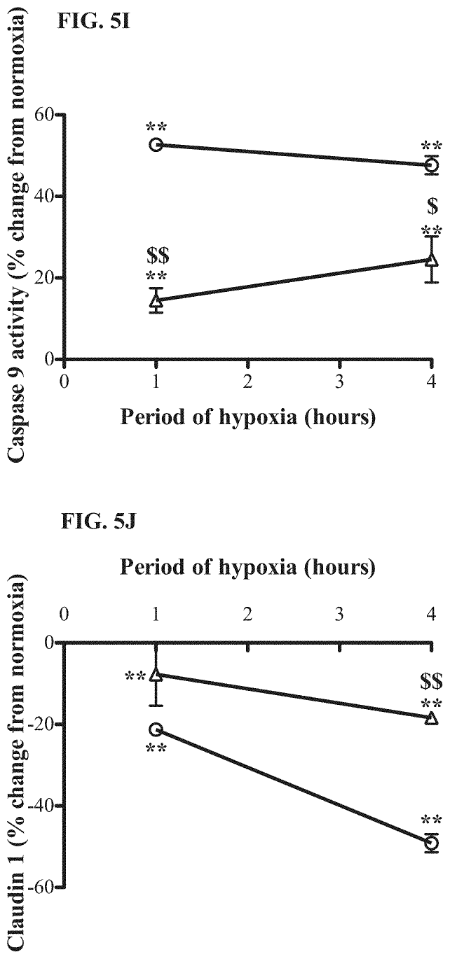

Cleared cell lysates from I/R experiments in Caco-2 cells were assessed for levels of the apoptotic molecules Caspase 3 (FIG. 5A), Caspase 9 (FIG. 5I), Baxα (FIG. 5B) and Bcl2 (FIG. 5C). HIF1α (FIG. 5Δ), VEGF (FIG. 5E), hSP70 (FIG. 5F), ICAM1 (FIG. 5G) and the tight junction molecules ZO1 (FIG. 5H) and Claudin1 (FIG. 5J) levels were also analysed.

Results

Apoptotic Pathways

PSTI pre-treatment had no effect on Caspase 3, 9, Baxα or Bcl2 under normoxic conditions in any of the cell lines (data not shown). Following H/R, the pro-apoptotic molecules Caspase 3 (FIG. 5A) and Caspase 9 (FIG. 5I) increased at both timepoints 1 hour and 4 hours. Pre-treatment with PSTI reduced this H/R-induced rise to by between 50 and 70% in both molecules (FIGS. 5A & I). Similarly, the pro-apoptotic Baxα increased in response to H/R but this rise was significantly truncated in the presence of PSTI (FIG. 5B). Conversely, the anti-apoptotic molecule Bcl2 was significantly reduced in response to H/R but this effect was truncated by the presence of PSTI (FIG. 5C).

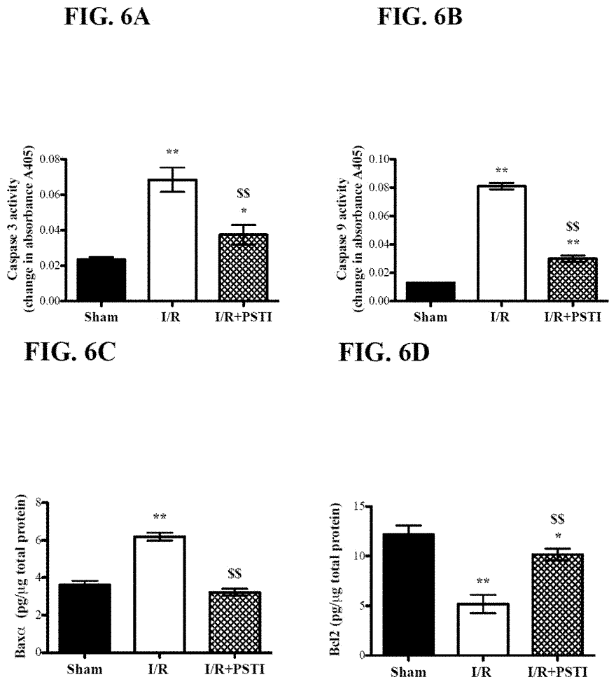

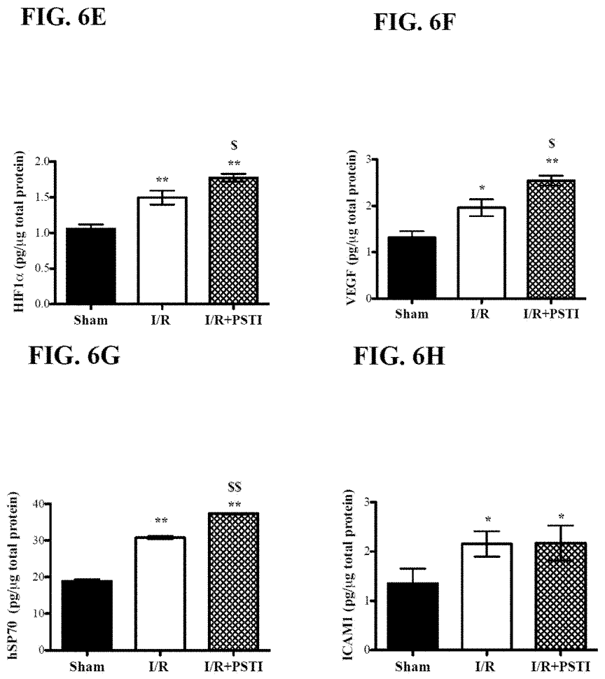

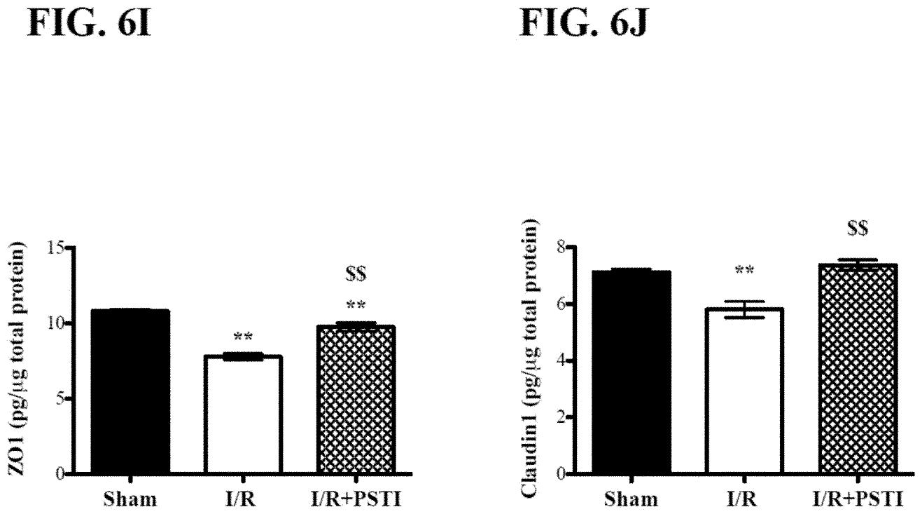

Similar results were seen in the animal study with Caspase 3 (FIG. 6A), Caspase 9 (FIG. 6B) and Baxα (FIG. 6C) all increased in I/R treated animals at the site of clamping (50% SI) compared to sham operated controls. These changes were reduced by between 70 and 100% in animals that had received PSTI. Reciprocal results were seen studying the anti-apoptotic peptide Bcl2 (FIG. 6D)

In the same animals, examination of the non-clamped region (90% SI) showed Caspase 3, 9 and Bcl2 did not change in the I/R group (Table 1). Distant organs (kidney, lung) showed no change in Caspase 3, 9, Baxα or Bcl2 in response to I/R (Tables 2 and 3, respectively).

TABLE 3
Effect of gut ischemia-reperfusion (I/R) +/− pre-administration
of PSTI on apoptotic and protective pathways in lung tissue in mice.

| Lung | Sham | I/R | IR + PSTI |
|---|---|---|---|
| Caspase 3 | 0.080 ± 0.020 | 0.075 ± 0.011 | 0.092 ± 0.022 |
| (Change in absorbance) | | | |
| Caspase 9 | 0.059 ± 0.005 | 0.060 ± 0.011 | 0.063 ± 0.004 |
| (Change in absorbance) | | | |
| Bcl2 | 44.11 ± 4.52 | 45.91± 4.91 | 45.66 ± 3.79 |
| (pg/μg total protein) | | | |
| Baxα | 3.17 ± 0.35 | 3.73 ± 0.24 | 3.74 ± 0.21 |
| (pg/μg total protein) | | | |
| HIF1α | 2.79 ± 0.43 | 2.66 ± 0.23 | 2.73 ± 0.48 |
| (pg/μg total protein) | | | |
| VEGF | 5.98 ± 0.71 | 5.81 ± 0.55 | 6.22 ± 0.98 |
| (pg/μg total protein) | | | |
| HSP70 | 20.95 ± 1.10 | 30.48 ± 0.81 ** | 39.73 ± 0.88 ** $$ |
| (pg/μg total protein) | | | |
| ICAM1 | 3.52 ± 0.53 | 5.93 ± 0.16 ** | 5.67 ± 0.13 ** |
| (pg/μg total protein) | | | |
| ZO1 | 8.46 ± 0.38 | 8.31 ± 0.51 | 8.15 ± 0.80 |
| (pg/μg total protein) | | | |
| Claudin 1 | 10.89 ± 1.23 | 8.83 ± 0.35 | 9.63 ± 0.40 |
| (pg/μg total protein) | | | |

Data presented as mean ±/− SEM.
** signifies p < 0.05 and <0.01 vs Sham operated animals (laparotomy only),
$$ signifies p < 0.05 and <0.01 vs I/R

HIF1α & VEGF

PSTI pre-treatment had no effect on HIF1α or VEGF, under normoxic conditions in any of the cell lines (data not shown). H/R induced a 42% increase in HIF1α after 4 h hypoxia+24 h reoxygenation time point and this was further increased by a further 11% in cells pre-treated with PSTI. (FIG. 5D). H/R caused an increase of VEGF levels, reaching 34% higher than normoxic values after 4 h hypoxia+24 h reoxygenation time point, with the co-presence of PSTI causing a further 15% increase (FIG. 5E).

Similar results were seen in the animal study with HIF1α and VEGF increasing at the clamped 50% SI site following I/R and pre-treatment with PSTI further increasing HIF1α and VEGF (FIGS. 6E&F). No changes in HIF1α and VEGF were seen in response to I/R+/−PSTI at the 90% SI site nor in the kidneys or lungs. (Tables 1, 2 and 3, respectively)

hSP70 & ICAM1

In cells grown under normoxic conditions throughout, PSTI caused a 46% increase in hSP70. H/R of cells caused a rise of about 63% at the 4 h hypoxia+24 h reoxygenation time point. The co-presence of PSTI resulting in an even greater increase of about 80% (FIG. 5F).

PSTI administered to cells under normoxic conditions had no effect on ICAM1 levels in any of the cell lines tested (data not shown). H/R caused a doubling in the level of ICAM1 after 4 h and was not influenced by the presence of PSTI (FIG. 5G)

Results from animal studies showed similar effects to that seen in cell studies, with I/R causing increased hSP70 expression in the 50% SI (clamped region), lungs and kidney, but not in the 90% SI (non-clamped region). Additional increases in hSP70 expression were found in the 50% SI, 90% SI, and in the lungs of animals that underwent I/R and also received PSTI (FIG. 6G, Table 1, Table 3, respectively)

ICAM1 was increased at the 50% SI site (FIG. 6H) and in the lungs (Table 3) in response to I/R but did not change in the kidney (Table 2) or 90% SI (Table 1). PSTI pre-treatment had no effect on these results.

Tight junctions:

H/R resulted in about 50% reduction in ZO1 (FIG. 5H) and Claudin1 (FIG. 5J) levels in the cell lines after 4 hours of hypoxia. The presence of PSTI truncated the fall in ZO1 and 1 by about 50%.

Animal studies showed similar results; ZO1 and Claudin1 decreased by about 20% in response to I/R at the site of clamping (50% SI) but this was virtually completely prevented in animals that had received the PSTI (FIGS. 6I & J, respectively). In contrast, no change in ZO1 or Claudin1 were found in non-clamped region of I/R treated animals (90% SI, Table 1), nor in the kidney (Table 2) or lung (Table 3).

In vitro studies examining effect of hypoxia for 1 or 4 h followed by reperfusion for 24 h using RIE1 cells showed PSTI reduced pro-apototic Caspase 3 and Caspase 9 and Baxα levels and normalised Bcl2 levels, caused additional increases in HIF1α, VEGF and Hsp70 levels above rises caused by I/R alone and prevented reduction of tight junction molecules ZO-1 and Claudin1 (all p<0.05), after 4 h hypoxia followed by 24 h reperfusion, as shown in Table 4. PSTI did not affect increased ICAM1.

TABLE 4
Effect of hypoxia-reperfusion (H/R) +/− PSTI on apoptotic and protective pathways in RIE1 cells

| | | Hyp 1 h/Rep | Hyp 1 h/Rep | Hyp 4 h/Rep | Hyp 4 h/Rep |
|---|---|---|---|---|---|
| RIE1 | Norm | 24 h | 24 h + PSTI | 24 h | 24 h + PSTI |
| Caspase 3 | 101.2 +/− 4.65 | 158.3 +/− 19.35 * | 112.3 +/− 2.70 * | 150.5 +/− 7.4 ** | 126.3 +/− 5.2 * $$ |
| (% normoxic) | | | | | |
| Caspase 9 | 99.4 +/− 4.22 | 180.5 +/− 3.95 ** | 120.6 +/− 17.02 * $ | 215.0 +/− 3.63 ** | 127.9 +/− 3.11 ** $$ |
| (% normoxic) | | | | | |
| Bcl2 | 99.6 +/− 0.24 | 51.4 +/− 0.58 ** | 49.3 +/− 1.91 ** $$ | 48.4 +/− 1.71 ** | 55.7 +/− 2.29 ** $$ |
| (% normoxic) | | | | | |
| Baxα | 102.1 +/− 1.15 | 115.7 +/− 1.27 ** | 97.6 +/− 0.01 ** $ | 133.5 +/− 0.07 ** | 115.6 +/− 1.73 ** $$ |
| (% normoxic) | | | | | |
| HIF1α | 102.3 +/− 0.42 | 130.7 +/− 1.76 ** | 144.1 +/− 0.17 ** $$ | 129.3 +/− 2.03 ** | 149.2 +/− 0.49 ** $$ |
| (% normoxic) | | | | | |
| VEGF | 101.5 +/− 1.11 | 111.7 +/− 1.46 ** | 121.8 +/− 2.53 ** $$ | 155.1 +/− 0.4188 | 174.9 +/− 0.003 ** $$ |
| (% normoxic) | | | | | |
| hSP70 | 97.7 +/− 2.68 | 119.7 +/− 1.98 ** | 122.9 +/− 0.85 ** | 150.5 +/− 5.6 ** | 171.1 +/− 3.97 ** $$ |
| (% normoxic) | | | | | |
| ICAM1 | 101.1 +/− 0.74 | 116.6 +/− 0.78 ** | 115.4 +/− 1.09 ** | 142.5 +/− 3.21 ** | 145.1 +/− 3.09 ** |
| (% normoxic) | | | | | |
| ZO1 | 100.5 +/− 4.68 | 73.0 +/− 3.0 ** | 83.6 +/− 0.69 * $ | 44.5 +/− 6.28 ** | 85.8 +/− 1.71 ** $$ |
| (% normoxic) | | | | | |
| Claudin1 | 100.5 +/− 2.09 | 56.4 +/− 3.1 ** | 75.6 +/− 2.1 ** $$ | 35.6 +/− 1.21 ** | 54.6 +/− 2.08 ** $$ |
| (% normoxic) | | | | | |

Data presented as mean +/− SEM.
** signifies p < 0.05 and <0.01 vs Sham operated animals (laparotomy only),
$$ signifies p < 0.05 and <0.01 vs H/R

In vitro studies examining effect of hypoxia for 1 or 4 h followed by reperfusion for 24 h using AGS cells showed PSTI reduced pro-apototic Caspase 3 and 9 and Baxα levels and normalised Bcl2 levels, caused additional increases in HIF1α, VEGF and Hsp70 levels above rises caused by I/R alone and prevented reduction of tight junction molecules ZO-1 and Claudin1 (all p<0.05), as shown in Table 5. PSTI did not affect increased ICAM1.

TABLE 5
Effect of hypoxia-reperfusion (H/R) +/− PSTI on apoptotic and protective pathways in AGS cells

| | | Hyp 1 h/Rep | Hyp 1 h/Rep | Hyp 4 h/Rep | Hyp 4 h/Rep |
|---|---|---|---|---|---|
| AGS | Norm | 24 h | 24 h + PSTI | 24 h | 24 h + PSTI |
| Caspase 3 | 100.9 +/− 2.21 | 287.1 +/− 9.34 ** | 228.2 +/− 18.72 * $ | 332.9 +/− 10.6 ** | 253.4.9 +/− 16.1 ** $$ |
| (% normoxic) | | | | | |
| Caspase 9 | 101.47 +/− 4.85 | 179.5 +/− 20.61 * | 119.8 +/− 13.25 * $ | 203.9 +/− 14.48 ** | 129.1 +/− 4.83 ** $$ |
| (% normoxic) | | | | | |
| Bcl2 | 103.9 +/− 0.83 | 42.2 +/− 1.42 ** | 49.3 +/− 1.91 ** $$ | 35.6 +/− 1.01 ** | 45.3 +/− 2.1 ** $$ |
| (% normoxic) | | | | | |
| Baxα | 101.4 +/− 1.22 | 124.9 +/− 4.0 * | 110.9 +/− 2.79 * $ | 146.2 +/− 2.46 ** | 131.6 +/− 0.03 ** $ |
| (% normoxic) | | | | | |
| HIF1α | 100.1 +/− 0.76 | 131.1 +/− 0.56 ** | 145.2 +/− 0.66 ** $$ | 139.3 +/− 2.03 ** | !61.2 +/− 1.01 ** $$ |
| (% normoxic) | | | | | |
| VEGF | 101.0 +/− 3.17 | 111.85 +/− 1.56 ** | 125.82 +/− 2.0 ** $$ | 154.2 +/− 1.09 ** | 167.9 +/− 1.31 ** $$ |
| (% normoxic) | | | | | |
| hSP70 | 98.6 +/− 2.01 | 129.7 +/− 1.76 ** | 132.9 +/− 1.05 ** $ | 160.5 +/− 4.2 ** | 178.9 +/− 1.05 ** $$ |
| (% normoxic) | | | | | |
| ICAM1 | 100.7 +/− 0.54 | 114.5 +/− 0.27 ** | 113.6 +/− 0.12 ** | 136.7 +/− 7.5 ** | 135.4 +/− 2.18 ** |
| (% normoxic) | | | | | |
| ZO1 | 101.1 +/− 1.12 | 74.5 +/− 2.13 ** | 82.1 +/− 0.98 ** $ | 45.7 +/− 3.21 ** | 84.7 +/− 1.06 ** $$ |
| (% normoxic) | | | | | |
| Claudin1 | 99.8 +/− 3.14 | 54.2 +/− 4.2 ** | 77.3 +/− 3.63 ** $$ | 34.5 +/− 2.2 ** | 55.4 +/− 0.68 ** $$ |
| (% normoxic) | | | | | |

Data presented as mean +/− SEM.
** signifies p < 0.05 and <0.01 vs Sham operated animals (laparotomy only),
$$ signifies p < 0.05 and <0.01 vs H/R.

In summary, H/R caused 50% drop in cell viability. PSTI (10 μg/ml) given prior- or during-hypoxic period improved survival by 50% (p<0.01). Caco-2 monolayers exposed to H/R had 300% increase in transepithelial permeability, PSTI truncated this by 50% (p<0.01). Mice that underwent mesenteric I/R had jejunal gut necrosis and 3-fold increases in MDA and MPO. Lung showed similar significant injury and inflammatory infiltrate markers. Smaller increases in MDA and MPO were seen in kidney. PSTI (20 μg/kg) reduced all injury markers by 50-80% (p<0.01). In vitro and in vivo studies showed PSTI reduced pro-apoptotic Caspase 3 and 9 and Baxα levels, normalised Bcl2 levels and caused additional increases in HIF1α, VEGF and Hsp70 above rises caused by I/R alone (all p<0.01). PSTI prevented reduction of tight junction molecules ZO1 and Claudin1 (all p<0.01) but did not affect increased ICAM1 caused by I/R in gut or lung.

AMINO ACID SEQUENCES

(SEQ ID NO: 1)
dslgreakcy nelngctkiy dpvcgtdgnt ypnecvlcfe nrkrqtsili qksgpc
(SEQ ID NO: 2)
mkvtgiflls alallslsgn tgadslgrea kcynelngct kiydpvcgtd gntypnecvl cfenrkrqts
iliqksgpc
(SEQ ID NO: 3)
dslgreakcy nelngctkiy dpvcgtdgdt ypnecvlcfe nrkrqtsili qksgpc
(SEQ ID NO: 4)
dslgreakcy nelngctrvy dpvcgtdgdt ypnecvlcfe nrkrqtsili qksgpc
(SEQ ID NO: 5)
dslgreakcy selngctkiy dpvcgtdgnt ypnecvlcfe nrkrqtsili qksgpc
(SEQ ID NO: 6)
dslgreakcy nelngctkiy dpvcgtdgnt ypnecvlcfe nqkrqtsili qksgpc
(SEQ ID NO: 7)
dslgreakcy nelngctkiy dpvcgtdgnt ypnecvlcfe nrksqtsili qksgpc
(SEQ ID NO: 8)
dslgreakcy nelngctkiy dpvcgtdgnt ypnecvlcfe nrtrqtsili qksgpc
(SEQ ID NO: 9)
mkvtgiflls alallslsgn tgadslgrea kcyselngct kiydpvcgtd gntypnecvl cfenrkrqts
iliqksgpc
(SEQ ID NO: 10)
dslgreakcy nelngctriy dpvcgtdgnt ypnecvlcfe nrkrqtsili qksgpc
(SEQ ID NO: 11)
dslgreakcy nelngcthiy dpvcgtdgnt ypnecvlcfe nrkrqtsili qksgpc
(SEQ ID NO: 12)
dslgreakcy nelngctkgy dpvcgtdgnt ypnecvlcfe nrkrqtsili qksgpc
(SEQ ID NO: 13)
dslgreakcy nelngctkay dpvcgtdgnt ypnecvlcfe nrkrqtsili qksgpc
(SEQ ID NO: 14)
dslgreakcy nelngctkly dpvcgtdgnt ypnecvlcfe nrkrqtsili qksgpc
(SEQ ID NO: 15)
dslgreakcy nelngctkvy dpvcgtdgnt ypnecvlcfe nrkrqtsili qksgpc
(SEQ ID NO: 16)
dslgreakcy nelngctkiy dpvcgtdgnt ypnecvlcfe nrkrqtsili qksgpc
(SEQ ID NO: 17)
dslgreakcy nelngctrgy dpvcgtdgnt ypnecvlcfe nrkrqtsili qksgpc
(SEQ ID NO: 18)
dslgreakcy nelngctray dpvcgtdgnt ypnecvlcfe nrkrqtsili qksgpc
(SEQ ID NO: 19)
dslgreakcy nelngctrly dpvcgtdgnt ypnecvlcfe nrkrqtsili qksgpc
(SEQ ID NO: 20)
dslgreakcy nelngctrvy dpvcgtdgnt ypnecvlcfe nrkrqtsili qksgpc
(SEQ ID NO: 21)
dslgreakcy nelngcthgy dpvcgtdgnt ypnecvlcfe nrkrqtsili qksgpc
(SEQ ID NO: 22)
dslgreakcy nelngcthay dpvcgtdgnt ypnecvlcfe nrkrqtsili qksgpc
(SEQ ID NO: 23)
dslgreakcy nelngcthly dpvcgtdgnt ypnecvlcfe nrkrqtsili qksgpc
(SEQ ID NO: 24)
dslgreakcy nelngcthvy dpvcgtdgnt ypnecvlcfe nrkrqtsili qksgpc

What is claimed is:

1. A method of treating, preventing, or alleviating a gastrointestinal ischemia/reperfusion-induced injury or an ischemia/reperfusion-associated condition in a subject in need thereof, comprising administering a composition comprising a therapeutically effective amount of an isolated human pancreatic secretory trypsin inhibitor peptide, a pharmaceutically acceptable salt thereof, or a prodrug thereof, and a pharmaceutically acceptable carrier.

2. The method of claim 1, wherein the ischemia/reperfusion-induced injury is selected from the group consisting of bacterial translocation, systemic inflammatory response syndrome, intestinal necrosis, intestinal transplant rejection, damage to remote organs, and remote organ failure.

3. The method of claim 1, wherein the ischemia/reperfusion-associated condition selected from the group consisting of necrotizing entercolitis, acute mesenteric ischemia, occlusion/infarction, trauma, transplantation, volvulus, cardiopulmonary disease, shock, acute vascular emergency, severe infectious colitis, non-occlusive mesenteric ischemia, ischemic colitis, and intestinal transplantation.

4. The method of claim 1, wherein the isolated human pancreatic secretory trypsin inhibitor (PSTI) peptide is a recombinant or synthetic pancreatic secretory trypsin inhibitor.

5. The method of claim 4, wherein the PSTI peptide comprises an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, or 24, ora substantially identical variant peptide, or an active peptide fragment or a functional homolog thereof.

6. The method of claim 5, wherein the PSTI peptide has an amino acid sequence of SEQ ID NO: 1.

7. The method of claim 5, wherein the substantially identical variant peptide shares at least 90%, at least 95%, at least 98% or at least 99% sequence identity with SEQ ID NO: 1.

8. The method of claim 5, wherein the active peptide fragment consists of at the most 55 consecutive amino acid residues, at the most 50 consecutive amino acid residues, at the most 40 consecutive amino acid residues, at the most 30 consecutive amino acid residues, from 15 to 30 consecutive amino acid residues, or from 18 to 25 consecutive amino acid residues of SEQ ID NO: 1, or a functional homolog thereof.

9. The method of claim 8, wherein the functional homolog has at the most three amino acid substitutions, two amino acid substitutions, or one amino acid substitution compared to the active peptide fragment.

10. The method of claim 1, wherein the therapeutically effective amount of the PSTI peptide, or pharmaceutically acceptable salt thereof, is from about 0.001 mg/kg to about 10 mg/kg, about 0.005 mg/kg to about 5 mg/kg, about 0.01 mg/kg to about 1 mg/kg, or from about 20 μg/kg to about 200 μg/kg body weight of the subject per day.

11. The method of claim 10, wherein the therapeutically effective amount of the PSTI peptide, or pharmaceutically acceptable salt thereof, is from about 0.01 mg to about 500 mg, about 0.05 mg to about 80 mg, about 0.1 to about 50 mg, about 0.5 to about 10 mg, or from about 1 mg to about 5 mg per day.

12. A method of reducing hypoxia-reoxygenation (H/R)-induced cell death comprising exposing the cell to a composition comprising a PSTI peptide, a pharmaceutically acceptable salt thereof, or a prodrug thereof, and a pharmaceutically acceptable carrier.

13. The method of claim 12, wherein the cells are mammalian gastrointestinal cells.

14. The method of claim 13, wherein the mammalian gastrointestinal cells are human stomach, small intestine, or large intestine cells.

15. The method of claim 14, wherein the small intestine cells are duodenum cells, jejunum cells, or ileum cells.

16. The method of claim 14, wherein the large intestine cells are cecum, colon, rectal, or anal cells.

17. A pharmaceutical composition for use in treating, preventing, or alleviating a gastrointestinal ischemia/reperfusion-induced injury or an ischemia/reperfusion-associated condition, the composition comprising a therapeutically effective amount of an isolated human pancreatic secretory trypsin inhibitor peptide, a pharmaceutically acceptable salt thereof, or a prodrug thereof, and a pharmaceutically acceptable carrier.

18. The composition of claim 17, wherein the PSTI peptide comprises an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, and 24, or a substantially identical variant peptide, or an active peptide fragment or a functional homolog thereof.

19. A pharmaceutical composition for use in reducing hypoxia-reoxygenation (H/R)-induced cell death comprising exposing the cell to a composition comprising a PSTI peptide, a pharmaceutically acceptable salt thereof, or a prodrug thereof, and a pharmaceutically acceptable carrier.

20. The composition of claim 19, wherein the PSTI peptide comprises an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, and 24, or a substantially identical variant peptide, or an active peptide fragment or a functional homolog thereof.
